SEQUENCE LISTING (15 sequences)

SEQ ID NO: 1 - protein, 12 aa, Artificial sequence
  Synthetic peptide
IDILLKAVGDTP

SEQ ID NO: 2 - protein, 140 aa, Homo sapiens
MAEEPQSVLQLPTSIAAGGEGLTDVSPETTTPEPPSSAAVSPGTEEPAGDTKKKIDILLKAVGDTPIMKTKKWAVERTRTIQGLIDFIKKFLKLVASEQLFIYVNQSFAPSPDQEVGTLYECFGSDGKLVLHYCKSQAWG

SEQ ID NO: 3 - protein, 12 aa, Artificial sequence
  Synthetic peptide
IDVLLKAVGDTP

SEQ ID NO: 4 - protein, 5 aa, Artificial sequence
  Synthetic peptide
KLVAS

SEQ ID NO: 5 - protein, 12 aa, Artificial sequence
  Synthetic peptide
IDVLLKAVGDDP

SEQ ID NO: 6 - protein, 12 aa, Artificial sequence
  Synthetic peptide
IDVLLKAVGDEP

SEQ ID NO: 7 - protein, 12 aa, Artificial sequence
  Synthetic peptide
IDILLKAVGDAP

SEQ ID NO: 8 - protein, 12 aa, Artificial sequence
  Synthetic peptide
IDILLKAVGDXP

SEQ ID NO: 9 - protein, 239 aa, Homo sapiens
MAHAGRTGYDNREIVMKYIHYKLSQRGYEWDAGDVGAAPPGAAPAPGIFSSQPGHTPHTAASRDPVARTSPLQTPAAPGAAAGPALSPVPPVVHLTLRQAGDDFSRRYRRDFAEMSRQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 10 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 11 - protein, 233 aa, Homo sapiens [sequence omitted]

SEQ ID NO: 12 - protein, 193 aa, Homo sapiens
MATPASAPDTRALVADFVGYKLRQKGYVCGAGPGEGPAADPLHQAMRAAGDEFETRFRRTFSDLAAQLHVTPGSAQQRFTQVSDELFQGGPNWGRLVAFFVFGAALCAESVNKEMEPLVGQVQEWMVAYLETRLADWIHSSGGWAEFTALYGDGALEEARRLREGNWASVRTVLTGAVALGALVTVGAFFASK

SEQ ID NO: 13 - protein, 204 aa, Homo sapiens
MVDQLRERTTMADPLRERTELLLADYLGYCAREPGTPEPAPSTPEAAVLRSAAARLRQIHRSFFSAYLGYPGNRFELVALMADSVLSDSPGPTWGRVVTLVTFAGTLLERGPLVTARWKKWGFQPRLKEQEGDVARDCQRLVALLSSRLMGQHRAWLQAQGGWDGFCHFFRTPFPLAFWRKQLVQAFLSCLLTTAFIYLWTRLL

SEQ ID NO: 14 - protein, 350 aa, Homo sapiens [sequence omitted]

SEQ ID NO: 15 - protein, 175 aa, Homo sapiens
MTDCEFGYIYRLAQDYLQCVLQIPQPGSGPSKTSRVLQNVAFSVQKEVEKNLKSCLDNVNVVSVDTARTLFNQVMEKEFEDGIINWGRIVTIFAFEGILIKKLLRQQIAPDVDTYKEISYFVAEFIMNNTGEWIRQNGGWENGFVKKFEPKSGWMTFLEVTGKICEMLSLLKQYC

(19) United States
(12) Patent Application Publication
(10) Pub. No.: US 2014/0155330 A1
(43) Pub. Date: Jun. 5, 2014

(54) Compositions And Methods For Modulating Apoptosis

(71) Applicant: Yeda Research and Development Co. Ltd., Rehovot (IL)

(72) Inventors: Adi Kimchi, Rehovot (IL); Assaf Rubinstein, Rehovot (IL)

(73) Assignee: YEDA RESEARCH AND DEVELOPMENT CO. LTD., Rehovot (IL)

(21) Appl. No.: 14/177,840

(22) Filed: Feb. 11, 2014

Related U.S. Application Data
(63) Continuation of application No. PCT/IL2012/050295, filed on Aug. 8, 2012.
(60) Provisional application No. 61/522,259, filed on Aug. 11, 2011.

(51) Int. Cl. C07K 14/47; G01N 33/566; G01N 33/574

(52) U.S. Cl. CPC C07K 14/4747; G01N 33/5748; G01N 33/566
USPC 514/18.9; 530/327; 435/375; 435/7.21; 435/6.1

(57) ABSTRACT

Peptides, compositions and methods for modulating apoptosis, useful for treating diseases or disorders characterized by dysregulated apoptosis, such as cancer or neurodegenerative diseases. Screening methods for identifying agents that mediate apoptosis by regulating Atg12 binding to Bcl-2 family members.

### FIELD OF THE INVENTION

The present invention relates to compositions and methods for modulating apoptosis useful for treating a disease or disorder characterized by dysregulated apoptosis, such as cancer and neurodegenerative diseases. The present invention further provides methods for identifying agents that mediate apoptosis by regulating Atg12 binding to Bcl-2 family members.

### BACKGROUND OF THE INVENTION

Autophagy is an evolutionarily conserved catabolic process in which cytoplasmic proteins and organelles are engulfed within de novo formed double-membrane vesicles (termed autophagosomes), and delivered to lysosomes for degradation and recycling. Autophagy promotes cell survival by clearance of damaged organelles and aggregate-prone proteins, elimination of intracellular pathogens, and recycling of essential macromolecules during periods of nutrient limitation. In specific cases, autophagy may also serve as a non-apoptotic form of programmed cell death through excessive self-consumption and/or selective degradation of pro-survival factors.

Autophagy is governed by a set of autophagy-related (Atg) genes that operate concertedly during the process of autophagosome biogenesis. Autophagy requires the function of two ubiquitin-like processes, which catalyze the covalent conjugation of Atg12 to Atg5, and of MAP1-LC3 (the mammalian orthologue of yeast Atg8) to the lipid phosphatidylethanolamine (PE). These reactions are facilitated by a common E1-like enzyme, Atg7, and by specific E2-like enzymes, Atg3 and Atg10, which subsequently catalyze the conjugation of LC3 to PE, and of Atg5 to the C-terminal glycine of Atg12, respectively. Finally, the Atg5-Atg12 conjugate proceeds to form an active multimeric complex together with Atg16 that localizes to sites of autophagosome assembly. Recently, Atg3 was reported as an additional conjugation partner of Atg12.

Whether promoting cell survival or cell death, autophagy is intimately linked with apoptosis, and the two processes engage in a complex and poorly understood molecular crosstalk. One level of crosstalk involves the utilization of common proteins that regulate both apoptosis and autophagy. Recently, data from several studies suggested that ‘core machinery’ proteins interplay between autophagy and apoptosis and act as essential components of one pathway and regulators of the other. Examples of such “dual-function” proteins include the apoptotic caspases, which suppress autophagy through cleavage of Beclin-1 and Vps34, and Atg5, which has apoptotic roles that are separate from its function in autophagy (Pyo et al., 2005; Yousefi et al., 2006; Zalckvar et al., 2010).

The human Bcl-2 family includes more than 30 anti- and pro-apoptotic molecules that bind to each other and form homo- and heterodimers. These proteins are characterized by the presence of one to four conserved Bcl-2 homology (BH) domains (BH1 to BH4). The antiapoptotic members include, inter alia, Bcl-2, Bcl-X_{L}, Bcl-w, Mcl-1, Bfl-1 (A1), and Boo (Diva). Both pro- and anti-apoptotic members of the B-cell lymphoma 2 (Bcl-2) family regulate autophagy in addition to their canonical function in controlling the intrinsic pathways of caspase activation. Bcl-2 and related anti-apoptotic family members suppress autophagy by binding to the Bcl-2 homology 3 (BH3) domain of Beclin-1, an essential component of the class III PI3K/Vps34 complex that is necessary for autophagy induction (Erlich et al., 2007; Germain et al., 2010; Pattingre et al., 2005; Sinha and Levine, 2008). Conversely, pro-apoptotic “BH3-only” proteins promote autophagy by disrupting the Bcl-2-Beclin-1 interaction.

Inhibiting apoptosis is widely accepted as a necessary step in the transition from normal to cancer cells, and several cancer therapies exert their effects by reversing this process. Commitment to apoptosis is caused by permeabilisation of the outer mitochondrial membrane—a process regulated by the binding between different members of the Bcl-2 family. Furthermore, Bcl-2 family members also bind to the endoplasmic reticulum, where they modify processes such as the unfolded-protein response and autophagy that also cause or modify different types of cell death.

Bcl-2 overexpression was initially described in follicular lymphomas as a consequence of a t(14; 18) translocation, and as a poor prognostic marker in acute myelogenous leukemia (AML) and non-Hodgkin's lymphomas. Overexpression of Bcl-2 was subsequently described in prostate, breast and colon carcinomas, as well as glioblastomas. Overexpression of Mcl-1, another antiapoptotic, Bcl-2-related protein, was identified in relapsed AML, and was associated with poor prognosis. Other changes in Bcl-2-related protein expression identified in cancer cells include different mutations in the Bax gene, and changes in the proapoptotic to antiapoptotic Bcl-2 protein ratio. With the growing understanding of the importance of the Bcl-2 family as crucial regulators of the decision to initiate apoptosis, much effort has been directed at developing molecules that modify function by directly binding to Bcl-2 proteins.

The inability of cancer cells to execute an apoptotic program due to defects in the normal apoptotic machinery is thus often associated with an increase in resistance to chemotherapy, radiation, or immunotherapy-induced apoptosis. Several cancer therapies, including chemotherapeutic agents, radiation, and immunotherapy, work by directly or indirectly inducing apoptosis in cancer cells. Primary or acquired resistance of human cancer of different origins to current treatment protocols due to apoptosis defects is a major problem in current cancer therapy.

Overexpression of Bcl-2 also prevents apoptosis induced by most and chemotherapeutic drugs, including alkylating agents, and topoisomerase inhibitors. Based on the role of Bcl-2 proteins in regulating mitochondrial membrane permeabilization, and their frequently modified expression in human cancers, Bcl-2 proteins are legitimate targets (antiapoptotic members) or prototypes (proapoptotic members) for inducing apoptosis either by themselves or in association with other anticancer drugs (Yves Pommier et al. 2004).

Targeting Bcl-2 family members for mediating apoptosis has been disclosed. Non limiting examples of such disclosures include International Publication No. WO 09/137,664 relating to antibodies specific to heterodimers of Bcl-2 family and uses thereof.

International Publication No. WO 08/021,211 relates to compounds for modulating apoptosis in cells over-expressing Bcl-2 family member proteins, pharmaceutical compositions containing these compounds, and methods of using the compounds for treating apoptosis-associated diseases such as, for example, neoplastic disease (e.g., cancer) or other proliferative diseases associated with the over-expression of a Bcl-2 family member protein.

International Publication No. WO 06/135985 provides therapeutic agents which inhibit pro-survival molecules and which are capable of inducing or facilitates apoptosis of a target cell or cell population such as cancer cells. WO 06/135985 further provides methods for generating or selecting the therapeutic molecules and pharmaceutical compositions comprising the therapeutic molecules.

International Publication No. WO 06/002474 provides alpha helical peptide mimetics that mimic BH3-only proteins, compositions containing them, their conjugation to cell-targeting moieties, and their use in the regulation of cell death.

None of the background art, however, discloses or suggests that Atg12 plays a role in apoptosis and that Atg12 derived peptides may be useful as pro-apoptotic peptides. Further, none of the background art discloses or suggests that agents which modulate Atg12 binding to anti-apoptotic Bcl-2 family members are useful in treating or ameliorating diseases or disorder characterized by dysregulated apoptosis, e.g., cell proliferative diseases or neurodegenerative diseases.

There exists a long-felt need effective means of inducing apoptosis in cancer cells thereby curing or ameliorating diseases such as cancer. Further, there exists a long-felt need for means of suppressing apoptosis thereby curing or ameliorating diseases such as neurodegenerative diseases. The development of novel agents capable of selectively regulating Atg12 binding to Bcl-2 anti-apoptotic family members, is therefore desirable.

### SUMMARY OF THE INVENTION

The present invention provides compositions and methods for regulating Atg12 mediated apoptotic activity. The compositions and methods of the invention are useful for treating diseases or disorders characterized by dysregulated apoptosis, including but not limiting to cancer and neurodegenerative diseases. The invention further provides Atg12 derived peptides, including but not limiting to peptides derived from the Atg12 BH3-like motif or the BH3-like proximal loop, useful in inducing cell apoptosis. The present invention further provides methods of identifying agents capable of regulating Atg12 mediated apoptosis.

It is disclosed for the first time that Atg12 is an essential regulator of mitochondrial apoptosis. Surprisingly, the apoptotic function of Atg12 is mediated by an interaction with anti-apoptotic members of the Bcl-2 family resulting in their inactivation. It is further disclosed herein that Atg12 comprises a BH3-like motif, which mediates Atg12 binding to Bcl-2 proteins. It is also disclosed herein that said binding is further mediated by a loop spatially adjacent to the BH3-like motif, the loop being formed by amino acids 93 to 97 of human Atg12. Surprisingly, the interaction between Atg12 and the Bcl-2 members occurs when Atg12 is un-conjugated as opposed to Atg12 conjugation (such as to Atg3 or Atg5) being an essential step in autophagy. Furthermore, the present invention demonstrates for the first time that p38 MAP kinase phosphorylates human Atg12 on Thr65, located within the BH3-like motif of Atg12. Without wishing to be bound by any theory or mechanism of action, phosphorylation of Thr65 enhances Atg12 binding to Bcl-2 proteins, resulting in apoptosis.

The present invention provides screening assays for identifying agents that modulate the binding of Atg12 to Bcl-2 anti-apoptotic family members thereby regulating apoptosis. According to some embodiments of the invention, said binding of Atg12 to a Bcl-2 anti-apoptotic protein occurs via a BH3-like motif within Atg12. In specific embodiments, the BH3-like motif is situated between amino acids 53-65 of human Atg12 (SEQ ID NO: 2). In a particular embodiment, the BH3-like motif has an amino acid sequence as set forth in SEQ ID NO: 1 (IDILLKAVGDTP) or SEQ ID NO: 3 (IDVLLKAVGDTP). According to additional embodiments of the invention, said binding of Atg12 to a Bcl-2 anti-apoptotic protein occurs via amino acids 93 to 97 (KLVAS; SEQ ID NO: 4) of human Atg12. According to another embodiment of the invention, said binding of Atg12 to a Bcl-2 anti-apoptotic protein occurs via the BH3 binding pocket of the Bcl-2 anti-apoptotic protein. A BH3 binding pocket of Bcl-2 anti-apoptotic proteins is known in the art as a hydrophobic groove formed by the BH1, BH2 and BH3 domains.

The present invention further provides compositions and methods for inducing Atg12 apoptotic activity, specifically by stimulating the binding of Atg12 to Bcl-2 anti-apoptotic family members, thereby being useful in treating or ameliorating pathologies such as cancer. In a specific embodiment, the present invention provides peptides derived from Atg12, or analogs or derivatives thereof, and pharmaceutical compositions comprising same, useful in inhibiting Bcl-2 anti-apoptotic peptides. In some embodiments, said peptide is derived from the BH3 like motif. In another embodiment, said peptide is derived from the loop proximal to BH3 like motif (e.g., amino acids 93 to 97 of human Atg12).

The present invention further provides compositions and methods for reducing Atg12 apoptotic activity, specifically by disrupting the binding of Atg12 to Bcl-2 anti-apoptotic family members, thereby being useful in treating or ameliorating pathologies characterized by excessive cell death such as neurodegenerative diseases.

In a certain embodiment of the invention, said Atg12 is a human Atg12. In a specific embodiment, human Atg12 has the amino acid sequence as set forth in SEQ ID NO: 2). In particular embodiments of the invention, Atg12 is un-conjugated. In yet another particular embodiment, Atg12 is conjugated. Non limiting examples of proteins which may be conjugated or un-conjugated to Atg12 include Atg5 or Atg3.

According to a first aspect, the present invention provides an isolated peptide of 5-30 amino acids, comprising the amino acid sequence as set forth in SEQ ID NO: 1 (IDILLKAVGDTP), or an analog or derivative thereof.

According to another embodiment, the peptide is of 5-25 amino acids. According to another embodiment, the peptide is of 5-20 amino acids. According to another embodiment, the peptide is of 5-15 amino acids. According to another embodiment, the peptide is about 12 amino acids long. According to a certain embodiment, the peptide consists of SEQ ID NO: 1.

According to another embodiment, the analog has at least 70% sequence identity to SEQ ID NO: 1. According to another embodiment, the analog has at least 80% sequence identity to SEQ ID NO: 1. According to another embodiment, the analog has at least 90% sequence identity to SEQ ID NO: 1. According to another embodiment, the analog has at least 95% sequence identity to SEQ ID NO: 1. According to a specific embodiment, said analog has the amino acid sequence of SEQ ID NO: 3 (IDVLLKAVGDTP).

In another embodiment, the analog comprises substitution of the threonine residue on position 11 with a phospho-mimicking residue. A “phospho-mimicking residue” as used herein refers to a residue which is not phosphorylated but displays physico-chemico properties similar to a residue carrying a phosphate ion (phosphorylated residue) such as for example aspartic acid or glutamic acid. In a particular embodiment, said analog has the amino acid sequence as set forth in SEQ ID NO: 5 (IDVLLKAVGDDP). In another particular embodiment, said analog has the amino acid sequence as set forth in SEQ ID NO: 6 (IDVLLKAVGDEP).

In a particular embodiment, the peptide binds a Bcl-2 anti-apoptotic protein. In yet another particular embodiment, the peptide binds the BH3 binding pocket of the Bcl-2 anti-apoptotic protein. In another embodiment, the Bcl-2 anti-apoptotic protein is a human Bcl-2 anti-apoptotic protein. According to particular embodiments of the invention, the Bcl-2 anti-apoptotic protein is selected from the group consisting of: Bcl-2, Bcl-X_{L}, Bcl-w, Bcl-B, Mcl-1 and A1 (Bfl-1). In a specific embodiment, said Bcl-2 anti-apoptotic protein is Mcl-1. In another specific embodiment, said Bcl-2 anti-apoptotic protein is Bcl-2.

According to some embodiments, the isolated peptide is a pro-apoptotic peptide. A “pro-apoptotic peptide” as used herein refers to the ability of the peptide to induce cell apoptosis. Without wishing to be bound by any theory or mechanism of action, said peptide of the invention targets and binds the BH3 binding pocket of the Bcl-2 anti-apoptotic protein, and mimics the binding of Atg12 to said Bcl-2 family members, thereby suppressing their activity and promoting cell apoptosis.

According to another aspect, the present invention provides a method of screening for a pro-apoptotic agent, comprising identifying an agent which enhances the binding of Atg12 to a Bcl-2 anti-apoptotic protein. According to some embodiments, the method comprises:
(a) exposing a cell expressing Atg12 and a Bcl-2 anti-apoptotic protein to a putative pro-apoptotic agent;
(b) determining the binding of Atg12 to the Bcl-2 anti-apoptotic protein;

wherein enhancement of said binding indicates that the agent is a pro-apoptotic agent.

According to another embodiment, the method further comprises step (c) determining the change in survival of the cell in the presence of the agent relative to a control.

According to specific embodiments, the putative agent is selected from the group consisting of: peptides, nucleic acids, organic molecules, inorganic compounds and antibodies or antigen binding fragments thereof.

According to some embodiments, the present invention provides a pro-apoptotic agent obtained by the screening method of the invention. According to another embodiment, the agent is an anti-cancer agent. According to another embodiment, the agent enhances Atg12 binding to a Bcl-2 anti-apoptotic protein. According to another embodiment, the agent binds the BH3-like domain within Atg12. According to another embodiment, the agent binds the BH3 binding pocket of the Bcl-2 anti-apoptotic protein. According to another embodiment, the agent binds Atg12 BH3-like domain and Bcl-2 BH3 binding pocket. In another embodiment, the agent binds the proximal loop adjacent to the BH3-like region of Atg 12.

According to additional embodiments, the present invention provides a pharmaceutical composition comprising said pro-apoptotic agent as an active ingredient, and a pharmaceutically acceptable carrier.

According to another aspect, the present invention provides a method for inducing apoptosis in a cell, the method comprises contacting the cell with the peptide of the invention or the pro-apoptotic agent obtained by the screening method of the invention. In one embodiment, said cell is a cancer cell. In another embodiment, the cancer cell is a chemoresistant cancer cell.

According to another aspect, the present invention provides a method for treating cancer in a subject in need thereof, comprising administering to the subject a therapeutically effective amount of a pharmaceutical composition comprising an isolated peptide of 5-30 amino acids comprising the amino acid sequence as set forth in SEQ ID NO: 1 (IDILLKAVGDTP), or an analog or derivative thereof, or the pro-apoptotic agent obtained by the pro-apoptotic screening method of the invention.

According to another embodiment, said method sensitizes the cancer cell to chemotherapy treatment. According to some embodiments of the invention, the method further comprises administering to the subject a chemotherapeutic agent. In some embodiment, the chemotherapeutic agent is an apoptosis-inducing chemotherapeutic agent. In some embodiments, the compositions and methods of the invention are useful in treating chemotherapeutic-resistant tumors. According to a particular embodiment, said cancer is a chemoresistant cancer.

According to certain embodiments, the cancer is a hematopoietic malignancy, such as lymphoma and leukemia. According to particular embodiments, the hematopoietic malignancy is selected from the group consisting of: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, and non-Hodgkin's lymphoma.

According to another embodiment, the cancer is a solid malignancy. According to particular embodiments, the solid malignancy is selected from the group consisting of: prostate cancer, breast cancer, skin cancer, colon cancer, lung cancer, pancreatic cancer, head and neck cancer, kidney cancer, ovarian cancer, cervix cancer, bone cancer, liver cancer, thyroid cancer and brain cancer.

According to some embodiments of the invention the subject is a mammal. According to particular embodiments, the subject is a human.

According to another aspect, the present invention provides an isolated peptide of 5-30 amino acids comprising the amino acid sequence as set forth in SEQ ID NO: 1 (IDILLKAVGDTP) or an analog or derivative thereof, or a pharmaceutical composition comprising same, for use in treating cancer in a subject in need thereof. In yet another aspect, the present invention provides use of said peptide or a pharmaceutical composition comprising same, for the preparation of a medicament for treating cancer in a subject in need thereof.

According to another aspect, the present invention provides an agent capable of enhancing the binding of Atg12 to a Bcl-2 anti-apoptotic protein for use in treating cancer in a subject in need thereof. In some embodiments, there is provided a pharmaceutical composition comprising said agent for use in treating cancer in a subject in need thereof. According to yet another aspect, the present invention provides use of an agent capable of enhancing the binding of Atg12 to a Bcl-2 anti-apoptotic protein for preparation of a medicament for treating cancer. In some embodiments, there is provided a pharmaceutical composition comprising the agent for preparation of a medicament for treating cancer. In one embodiment, said agent is the pro-apoptotic agent obtained by the pro-apoptotic screening method of the invention.

According to another aspect, the present invention provides a method of screening for an anti-apoptotic agent, comprising identifying an agent which disrupts (e.g. reduce or interfere with) the binding of Atg12 to a Bcl-2 anti-apoptotic protein. According to a certain embodiment, the method comprises:
(a) exposing a cell expressing Atg12 and a Bcl-2 anti-apoptotic protein to a putative anti-apoptotic agent;
(b) determining the binding of Atg12 to the Bcl-2 anti-apoptotic protein;

wherein reduction of said binding indicates that the agent is an anti-apoptotic agent.

According to another embodiment, the method further comprises step (c) determining the change in survival of the cell in the presence of the agent relative to a control. According to yet another embodiment, said binding of Atg12 to the Bcl-2 anti-apoptotic protein is mediated by phosphorylation of the threonine residue in position 65 of Atg12. Non limiting examples of anti-apoptotic agents, according to specific embodiments of the invention, include agents capable of disrupting the phosphorylation of the threonine residue in position 65 of Atg12.

According to specific embodiments, the putative agent is selected from the group consisting of: peptides, nucleic acids, organic molecules, inorganic compounds and antibodies or antigen binding fragments thereof.

According to some embodiments, the present invention provides an anti-apoptotic agent obtained by the anti-apoptotic screening method of the invention. According to another embodiment, the agent reduces Atg12 binding to a Bcl-2 anti-apoptotic protein. According to another embodiment, the agent binds the BH3-like domain within Atg12. According to another embodiment, the agent binds the BH3 binding pocket of the Bcl-2 anti-apoptotic protein. In another embodiment, the agent binds the proximal loop adjacent to the BH3-like region of Atg12. In a specific embodiment, said loop has the amino acid sequence as set forth in SEQ ID NO: 4 (KLVAS).

According to additional embodiments, the present invention provides a pharmaceutical composition comprising said anti-apoptotic agent as an active ingredient, and a pharmaceutically acceptable carrier.

According to another aspect, there is provided an analog or derivative of SEQ ID NO: 1, wherein the residue in position 11 is a phospho-silencing residue. A “phospho-silencing residue” as used herein refers to a residue which is incapable of phosphorylation and is other than a phospho-mimicking residue. In some embodiments, the present invention provides an isolated peptide of 5-30 amino acids, comprising the amino acid sequence IDILLKAVGDXP wherein X is selected from the group consisting of alanine, isoleucine, leucine, asparagine, lysine, methionine, phenylalanine, glutamine, tryptophan, glycine, valine, proline, arginine and histidine (SEQ ID NO: 8). Each possibility represents a separate embodiment of the invention. According to one embodiment, said peptide comprises the amino acid sequence IDILLKAVGDAP (SEQ ID NO: 7).

According to another embodiment, the analog has at least 70%, at least 80% or at least 90% sequence identity to SEQ ID NO: 6. According to another embodiment, the peptide is of 5-25 amino acids, 5-20 amino acids or 5-15 amino acids.

According to another aspect, the present invention provides a method for treating a disease or disorder characterized by excessive cellular death in a subject in need thereof, comprising administering to the subject a therapeutically effective amount of a pharmaceutical composition comprising the anti-apoptotic agent obtained by the anti-apoptotic screening method of the invention.

According to some embodiments, the disease or disorder characterized by excessive cellular death is a neurodegenerative disease. According to another embodiment the neurodegenerative disease is selected from the group consisting of: Alzheimer's disease, Huntington's disease, Parkinson's disease, neurodegeneration due to stroke, amyotrophic lateral sclerosis (ALS), Pick's disease, Progressive Supranuclear Palsy (PSP), fronto-temporal dementia (FTD), pallido-ponto-nigral degeneration (PPND), Guam-ALS syndrome, pallido-nigro-luysian degeneration (PNLD) and cortico-basal degeneration (CBD).

According to another embodiment, the disease or disorder is associated with neuronal cell death. In specific embodiments, the disease or disorder associated with neuronal cell death is selected form the group consisting of: epilepsy, hypoxia/ischemia related acute brain injury, Parkinson's disease and Alzheimer's disease.

According to another aspect, the present invention provides the anti-apoptotic agent obtained by the screening method of the invention, or a pharmaceutical composition comprising same, for use in treating a disease or disorder characterized by excessive cellular death in a subject in need thereof.

In yet another aspect, the present invention provides use of the anti-apoptotic agent obtained by the screening method of the invention, or a pharmaceutical composition comprising same, for the preparation of a medicament for treating a disease or disorder characterized by excessive cellular death in a subject in need thereof.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description

### BRIEF DESCRIPTION OF THE DRAWINGS

FIGS. 1A-1D depicts an siRNA-based screen of mammalian autophagy genes to identify positive mediators of apoptosis. (A) HEK293 cells transfected with the indicated siRNA pools were treated with 50 μM etoposide for 48 h. For each gene, levels of caspase-3/7 activity relative to the average of three non-targeting controls were determined using the Caspase-Glo 3/7 assay. Results represent mean±SD of combined data from three independent screens. (B) HEK293 cells transfected with indicated siRNA pools were treated with 50 μM etoposide for 48 h and subjected to western blot analysis for molecular markers of apoptosis. (C) Caspase-3/7 activity relative to non-targeting siRNA pool in HeLa cells treated with STS (2 μM; 4 h), paclitaxel (10 μM; 24 h), etoposide (100 μM; 24 h), TNF-α/CHX (60 ng/ml; 5 h) and tunicamycin (1.5 μg/ml; 24 h); and in A549 cells treated with C₆-ceramide (50 μM; 24 h), etoposide (100 μM; 24 h) and STS (2 μM; 4 h). Non-treated cells were transfected with non-targeting siRNA pool. (D) ACHN cells transfected with the indicated siRNAs were treated with 200 ng/ml of anti-CD95 agonistic Ab (CH-11) for 5 h. Relative caspase-3/7 activity was determined using the Caspase-Glo 3/7 assay.

FIGS. 2A-2D shows a correlation between pairs of biological replicates of the screen (S1-S3) (A). Normalized data were plotted for each pair of biological replicates. Each point represents a single targeted gene. (B) Quantitation of cell number in HEK293 cells transfected with the indicated siRNA pools using the CyQUANT NF assay. (C, D) HEK293 cells transfected with the indicated siRNA pools (C) or individual siRNA duplexes (D) were treated with 50 μM etoposide for 48 h. For each gene, levels of caspase-3/7 activity relative to the average of two non-targeting controls were determined using the Caspase-Glo 3/7 assay.

FIGS. 3A-3E shows depletion of Atg12 effect in increasing survival of cells undergoing apoptotic cell death. (A) HeLa cells transfected with the indicated siRNAs were treated with STS (2 μM; 4 h) and subjected to western blot analysis for molecular markers of apoptosis. Arrow indicates p85 cleaved form of PARP-1. (B) STS-treated HeLa cells (0.5 μM; 8 h) were fixed and stained with DAPI. Cells exhibiting nuclear fragmentation were counted under a fluorescent microscope. At least 200 cells were counted per sample in triplicates. Results represent mean±SD of combined data from three independent experiments. (C) HeLa cells transfected with the indicated siRNAs were treated with various concentrations of STS for 12 h. The percent of PI-positive cells was determined by flow cytometry. (D) HeLa cells transfected with the indicated siRNAs were treated with 0.5 μM STS for 12 h. Cell viability was assessed using the XTT colorimetric assay. Non-treated cells were transfected with non-targeting siRNA. (E) HeLa cells transfected with the indicated siRNAs were treated with 0.5 μM STS. After 12 h, cells were trypsinized, re-plated and incubated for 5 days in fresh medium. Surviving cells were fixed and stained with crystal violet dye.

FIG. 4 depicts the effect of various inducers of apoptosis on HeLa and A549 cells transfected with the indicated siRNAs. Cell viability was determined using the XTT assay.

FIGS. 5A-5H shows Atg12 interaction with Bcl-2-related proteins via a BH3-like motif. (A) Sequence alignment of the BH3-like region of Atg12 with BH3 domains of the Bcl-2 family using ClustalW. The four conserved BH3 hydrophobic residues are colored in grey, and the conserved “GD” residues in black. Arrow indicates critical Asp64 residue in hAtg12. Species abbreviations: Hs, Homo sapiens; Sc, Saccharomyces cerevisiae; Dm, Drosophila melanogaster. (B) Co-immunoprecipitation of Flag-tagged hAtg12 and Bcl-2 or Mcl-1 from HEK293 cells co-transfected as indicated. (C) Co-immunoprecipitation of Flag-tagged hAtg12 with endogenous Bcl-2 and Mcl-1 from HEK293 cells. (D) Co-immunoprecipitation of endogenous Atg12 and endogenous Mcl-1 in HEK293 and HeLa cells. (E) Co-immunoprecipitation of Flag-tagged Atg12 and endogenous Bcl-2 in HeLa cells treated with STS (3 μM; 4 h). (F) Co-immunoprecipitation of Flag-tagged Atg12 and endogenous Bcl-2 in HEK293 cells co-expressing a control plasmid (CAT) or HA-tagged BAD. (G) Flag-Atg12 was immunoprecipitated from HEK293 cells and incubated with either 10 μM ABT-737 or DMSO (vehicle control). Co-precipitating Bcl-2 was assessed by western blot after elution with excess Flag peptide. (H) Co-immunoprecipitation of wild-type Atg12 or Atg12/D64 mutants with Bcl-2 in HEK293 cells co-transfected as indicated. In B, C, F-H, DAP1-Flag was used instead of Flag-Atg12 as a negative control for co-immunoprecipitation.

FIGS. 6A-6B (A) shows a multiple sequence alignment of Atg12 orthologues; alignment was performed using ClustalW and displayed using GeneDoc. (B) coimmunoprecipitation of Flag-tagged Bcl-2 and Atg12 from HEK293 cells co-transfected as indicated. DAP1-Flag was used as a negative control.

FIGS. 7A-7B shows that Atg12 binding to Bcl-2 does not require conjugation activity. (A) Co-immunoprecipitation of endogenous Atg12 in HEK293 cells expressing FLAG-tagged Bcl-2. DAP1-Flag was used as a negative control. Asterisk indicates non-specific band. (B) Co-immunoprecipitation of wild-type Atg12 or a conjugation-defective (ΔG140) mutant with Bcl-2 in transiently transfected HEK293 cells. DAP1-Flag was used as a negative control.

FIGS. 8A-8D shows that Bcl-2 interacts with Atg12, but not with Atg5. (A) HEK293 cells were co-transfected with Flag-tagged Bcl-2, HA-Atg5 and Atg12. Bcl-2 co-immunoprecipitated with Atg12, but not with Atg5 or the Atg5-Atg12 conjugate. (B) Expression levels of autophagy proteins and Bcl-2-related proteins in HeLa cells transfected with the indicated siRNA pools. (C) Expression levels of Bcl-xL and Mcl-1 in HEK293 cells transfected with either non-targeting control (siCtrl), or five different siRNA duplexes targeting Atg3. (D) Relative caspase-3/7 activity in etoposide-treated HEK293 cells transfected with the indicated siRNAs.

FIGS. 9A-9C is an In silico model of Atg12-Mcl-1 interaction. (A) The predicted mode of interaction between Atg12 and Mcl-1 based on protein-protein docking. Atg12 is depicted by a bright ribbon diagram. Only the side chains of residues D64 and V95 are shown as space filling spheres, colored by atom type (bright for C and dark for O). Mcl-1 is shown in cyan with R263 emphasized in blue and D256 in red. Top panel: two views from 90° rotation about the vertical axis. Bottom panel: a close-up view of the hydrophobic cleft region of Mcl-1. (B) Co-immunoprecipitation of WT Atg12 or Atg12/V95A mutant with Mcl-1 in transiently transfected HEK293 cells. (C) Co-immunoprecipitation of Atg12 with WT Mcl-1 or Mcl-1/R263A mutant in transiently transfected HEK293 cells.

FIG. 10 depicts co-immunoprecipitation of Flag-tagged WT Atg12 or a mutant of Atg12 (F101A) which is not implicated in Mcl-1 binding. Atg12/V95A mutation was used as positive control for mutation that affects Atg12-Mcl-1 binding.

FIGS. 11A-11E shows that Atg12 functions upstream of MOMP. (A, B) Relative caspase-3/7 activity in STS-treated HeLa cells (2 μM; 4 h) transfected with increasing amounts of Mcl-1 plasmid together with: (A) CAT (control), WT Atg12, or Atg12/V95A mutant; or with: (B) CAT (control), WT Atg12, or ΔG140 mutant. Results represent mean±SD of combined data from three independent experiments. (C) Immunoprecipitation of conformationally active Bax from STS-treated HeLa cells transfected with the indicated siRNAs. (D) HeLa cells were co-transfected with the indicated siRNAs together with plasmids encoding for either CAT (control) or siRNA-resistant Atg12. 48 h post-transfection, Active Bax was immunoprecipitated following treatment with STS (2 μM; 4 h). N.T, non-treated. (E) HeLa cells transfected with the indicated siRNAs were treated with STS (2 μM; 4 h), and subjected to sub-cellular fractionation by differential centrifugation. Left panel: representative fractionation experiment. Tubulin was used as cytosolic (soluble fraction) marker. Purity of cytosolic fractions was verified by blotting with a mitochondrial marker (Grp-75). Right panel: quantitation of cytosolic cytochrome c levels following siRNA-mediated depletion of Atg12. Results represent mean±SD of combined data from three independent fractionation experiments. Asterisk denotes p<0.05.

FIGS. 12A-12B depicts Bcl-2 binding-defective mutants of Atg12 retaining the autophagic activity. (A) Top panel: representative images of GFP-LC3 staining in Atg12-depleted HEK293 cells undergoing starvation (EBSS; 4 h). Cells were reconstituted with siRNA-resistant constructs expressing Flag-tagged wild-type Atg12 or the Bcl-2 binding-defective mutants (D64N/S). Bottom panel: quantitation of GFP-LC3 puncta per total cell area. Data represent mean±SD of triplicate measurements of at least 200 cells each. (B) Western blot analysis displaying reconstitution of the Atg5-Atg12 conjugate in cell lysates taken from GFP-LC3 HEK293 cells analyzed in (A). Specific autophagic degradation of p62 serves as indication of autophagic flux.

FIG. 13 depicts co-immunoprecipitation of Flag-tagged Atg12 and HA-tagged p38 in HEK293 cells.

FIGS. 14A-14B depicts a radioactive kinase assay. (A) Phosphorylated Atg12 is indicated by an arrow. The radioactive band above Atg12 is phosphorylated (active) p38. (B) Gel code staining showing equal loading of recombinant proteins.

FIG. 15 shows a multiple sequence alignment of BH3-only proteins. Most of the proteins contain a negatively charged residue (E/D) in the site corresponding with Thr65 of human Atg12.

### DETAILED DESCRIPTION OF THE INVENTION

The present invention provides screening assays for identifying agents capable of modulating (e.g., disrupting or enhancing) the binding of Atg12 to Bcl-2 anti-apoptotic family members, and therefore, treat pathologies characterized by dysregulated apoptosis. The present invention further provides compositions and methods for regulating Atg12 mediated apoptotic activity, useful for treating diseases and disorders such as proliferative disease or neurodegenerative diseases. In particular embodiments, the present invention provides an isolated peptide corresponding to Atg12 BH3-like domain and pharmaceutical composition comprising said peptide, useful in treating pathologies such as proliferative diseases.

As exemplified herein below, Atg12 plays a role in inactivating anti-apoptotic members of the Bcl-2 family, thus acting as an essential regulator of mitochondrial apoptosis. It is further exemplified that Atg12 comprises a BH3-like motif (situated between amino acids 53-65 of human Atg12) required for the binding to the Bcl-2 proteins. It is further exemplified that Atg12 comprises a loop proximal to the BH3 like motif which also mediates Atg12 binding to Bcl-2 proteins. In specific embodiments, the loop is situated between (or is formed by) amino acids 93-97 (KLVAS set forth as SEQ ID NO: 4) of human Atg12.

Without wishing to be bound by any theory or mechanism of action, the compositions and methods of the invention mediate apoptosis by targeting the binding of Atg12 to Bcl-2 family members, specifically via the newly identified BH3-like domain.

Thus, the present invention provides screening assays for identifying agents that modulate the binding of Atg12 to Bcl-2 family members thereby regulating apoptosis. The present invention further provides compositions and methods for enhancing Atg12 apoptotic activity, such as by enhancing Atg12 binding to Bcl-2 anti-apoptotic family members, thereby being useful in treating or ameliorating diseases such as cancer. In a specific embodiment of the invention, the compositions and methods are particularly useful in treating chemotherapeutic-resistant tumors. The present invention further provides compositions and methods for reducing Atg12 apoptotic activity, such as by disrupting Atg12 binding to Bcl-2 anti-apoptotic family members, thereby being useful in treating or ameliorating diseases characterized by excessive cellular death.

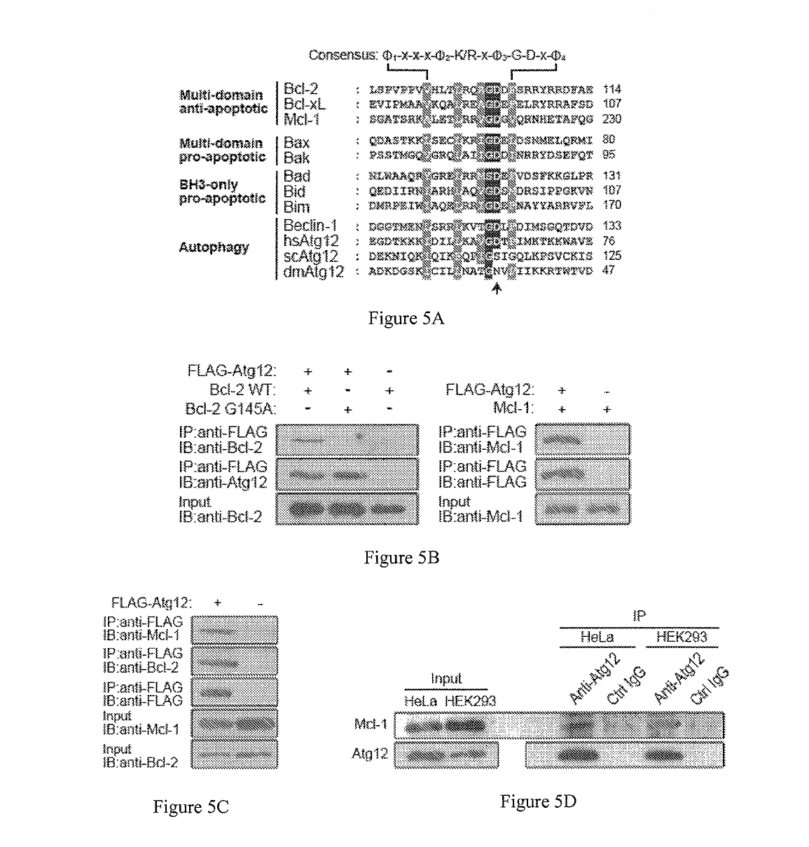

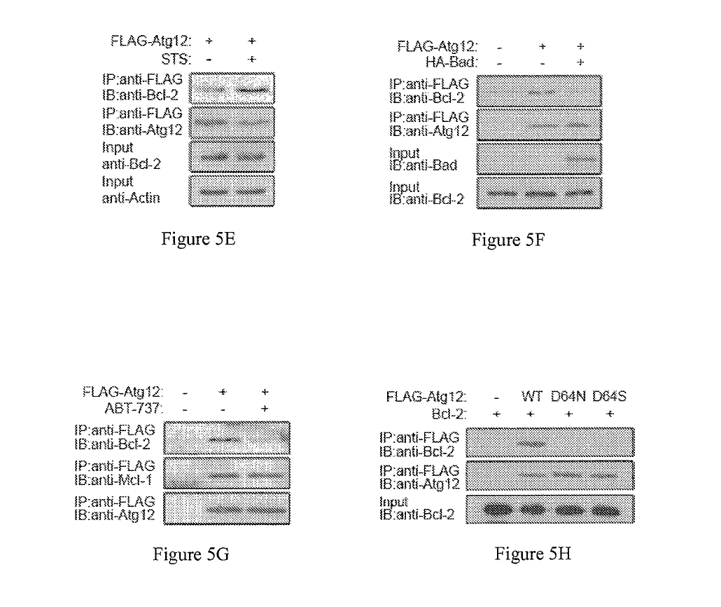

FIG. 5A depicts a sequence alignment of the BH3-like region of human Atg12 with BH3 domains of several Bcl-2 family members. The identified BH3-like motif is about 12 amino acid long and in particular embodiments resides between amino acid 55-66 of human Atg12 (SEQ ID NO: 2; NP_{—}004698.3). In particular embodiments said BH3-like domain has the amino acid sequence as set forth as SEQ ID NO: 1 (IDILLKAVGDTP). In another embodiment said BH3-like domain has the amino acid sequence as set forth as SEQ ID NO: 3 (IDVLLKAVGDTP).

As exemplified herein below, phosphorylation of the threonine residue in position 65 of Atg12 (i.e., the threonine in position 11 of SEQ ID NO: 1 or 3) enhances Atg12 interaction with Bcl-2 proteins. In some embodiments, the present invention provides analogs of SEQ ID NO: 1 (IDILLKAVGDTP), comprising an altered phosphorylation site in position 11 of SEQ ID NO: 1. The term “altered phosphorylation site” as used herein refers to an alteration of a phosphorylation site by an amino acid substitution and/or by chemical modification.

In other embodiments, the altered phosphorylation site relates to the threonine residue substituted with a phospho-mimicking residue. According to some embodiments, a peptide comprising substitution of said threonine residue with a phospho-mimicking residue (such as, aspartic acid or glutamic acid), binds Bcl-2 anti-apoptotic proteins, thus acting as a pro-apoptotic peptide. In a particular embodiment, said peptide comprises or consists of the amino acid sequence selected from SEQ ID NO: 5 (IDVLLKAVGDDP) and SEQ ID NO: 6 (IDVLLKAVGDEP).

In some embodiments, the altered phosphorylation site relates to the threonine residue substituted with a phospho-silencing residue. According to some embodiments, a peptide comprising substitution of said threonine residue with a phospho-silencing residue is useful as an anti-apoptotic peptide. In some embodiments, the peptide comprises or consists of the amino acid sequence as set forth in SEQ ID NO: 6. In another embodiment, said peptide comprises or consists of the amino acid sequence IDILLKAVGDAP (SEQ ID NO: 7).

The term “phosphorylation” has the meaning known in the art, e.g., the term refers to a phosphate transfer in which a phosphate group from a donor molecule is transferred to an acceptor molecule. Typically, phosphorylation usually occurs on serine, threonine, and tyrosine residues in eukaryotic proteins. Under cellular conditions phosphorylation is achieved enzymatically by an enzyme such as a kinase. Specifically, the term refers to the chemical addition of a phosphate group (e.g., PO₄²-) to Atg12. The present invention particularly relates to the phosphorylation on threonine residues, and specifically to Thr65 of human Atg12 (i.e., threonine in position 11 of the BH3-like region of Atg12 (SEQ ID NO:1)).

In some embodiments, the methods of the invention comprise altering the phosphorylation of Atg12. The phrase “altering the phosphorylation of Atg12” includes enhancing Atg12 phosphorylation and reducing or inhibiting Atg12 phosphorylation, particularly on Thr65 of human Atg12. The term “reducing or inhibiting Atg12 phosphorylation” as used herein includes preventing phosphorylation of Thr65 of human Atg12. This term also includes decreasing the extent of phosphorylation of Atg12 by preventing phosphorylation occurring at said phosphorylation site, or as a result of dephosphorylation occurring at said phosphorylated site.

Techniques are well known in the art for analyzing phosphorylation modification states. For example, phosphorylation may be determined by the use of antibodies to phospho-epitopes to detect a phosphorylated polypeptide by Western analysis, for example, as described in the Examples herein.

A “phospho-silencing residue” as used herein refers to a residue which is incapable of phosphorylation (e.g., a nonphosphorylatable residue) and is other than a phospho-mimicking residue. According to one embodiment, the phospho-silencing residue is selected from the group consisting of alanine, isoleucine, leucine, asparagines, lysine, methionine, phenylalanine, glutamine, tryptophan, glycine, valine, proline, arginine and histidine. According to some embodiments, the phospho-silencing residue is alanine.

A “phospho-mimicking residue” as used herein refers to a residue which is not phosphorylated but displays physico-chemico properties similar to a residue carrying a phosphate ion (phosphorylated residue) such as for example aspartic acid or glutamic acid. According to one embodiment, the phospho-mimicking residue is negatively charged. According to another embodiment the phospho-mimicking residue is negatively charged at pH above the pl of the phospho-mimicking residue. According to another embodiment, the phospho-mimicking residue is negatively charged at physiological pH (pH=7.4).

In some embodiment, the agents and/or peptides of the present invention are capable of binding the BH3 like domain of Atg12 and consequently modulate Atg12 apoptotic activity. In other embodiments, the agents and/or peptides of the present invention mimic the BH3 like domain of Atg12 and bind the BH3 binding pocket of Bcl-2 anti-apoptotic proteins. In another embodiment, the agent and/or peptides bind the proximal loop adjacent to the BH3 like region of Atg12. In a specific embodiment, said loop has the amino acid sequence as set forth in SEQ ID NO: 4 (KLVAS).

The term “modulate Atg12 apoptotic activity” as used herein refers to the activation (e.g., enhancement) or reduction (e.g., inhibition) of Atg12 pro-apoptotic activity, such as by targeting Atg12 interaction with a Bcl-2 anti-apoptotic family member. Modulating Atg12 apoptotic activity, in some embodiment, relates to promoting or inducing apoptosis. In some embodiments, modulating Atg12 apoptotic activity relates to inhibiting or reducing cell apoptosis. Methods for assaying both anti-apoptotic and pro-apoptotic activities are well known in the art and described herein.

As used herein, the term “interacts” or “binds” refers to a condition of proximity between two compounds, or portions thereof. A non limiting example is the binding of Atg12 BH3 like domain with a BH3 binding pocket (or groove) of a Bcl-2 anti-apoptotic family members. In some embodiment, the association may be non-covalent wherein the juxtaposition is energetically favored by hydrogen bonding or van der Waals or electrostatic interactions. In some embodiment, the association is covalent.

In another embodiment, said agents disrupt (e.g. reduce) the binding of Atg12 to anti-apoptotic Bcl-2 family members. Reducing or disrupting the binding of Atg12 to a Bcl-2 anti-apoptotic protein as used herein refers to a decrease in the association of Atg12 and a Bcl-2 anti-apoptotic protein, e.g., via the BH3-like domain within Atg12. This can include but is not limited to the complete disruption of the binding, or alternatively, at least a 10% reduction of Atg12 binding to Bcl-2 members as compared to the control (e.g., without the putative agent). Thus, the reduction can be a 10, 20, 30, 40, 50, 60, 70, 80, 90, 100%, or any amount of reduction in between the specifically recited percentages, as compared to native or control levels. In specific embodiments, an agent which reduces Atg12 binding to anti-apoptotic Bcl-2 family members is an anti-apoptotic agent. An anti-apoptotic agent reduces apoptosis, e.g., cell death, of at least 90%, 85%, 80%, 75%, 70%, 60%, 50%, 40%, 30%, 20%, 10%, 5% in a population of cells in which the anti-apoptotic agent is present than compared to a control cell population where the anti-apoptotic agent is not present.

In another embodiment, said agent enhances or promotes the binding of Atg12 to anti-apoptotic Bcl-2 family members. Enhancing or promoting the binding of Atg12 to a Bcl-2 anti-apoptotic protein as used herein refers to an elevation in the association of Atg12 and a Bcl-2 anti-apoptotic protein, e.g., via the BH3-like domain within Atg12. This can include but is not limited to the at least a 10% elevation of Atg12 binding to Bcl-2 members as compared to the control (e.g., without the putative agent). Thus, the elevation can be a 10, 20, 30, 40, 50, 60, 70, 80, 90, 100%, or any amount of elevation in between the specifically recited percentages, as compared to native or control levels. In specific embodiments, an agent which promotes Atg12 binding to anti-apoptotic Bcl-2 family members is a pro-apoptotic agent. A pro-apoptotic agent elevates apoptosis, e.g., cell death, of at least 90%, 85%, 80%, 75%, 70%, 60%, 50%, 40%, 30%, 20%, 10%, 5% in a population of cells in which the pro-apoptotic agent is present than compared to a control cell population where the pro-apoptotic agent is not present.

Methods to determine association of two proteins (e.g., binding of Atg12 and a Bcl-2 anti-apoptotic protein) are well known in the art, such as co-immunoprecipitation (IP).

Bcl-2 anti-apoptotic family members are well known in the art. In one embodiment, Bcl-2 has the amino acid sequence as set forth in SEQ ID NO: 9 (Accession No. AAA35591). In another embodiment, Bcl-2 has the amino acid sequence as set forth in SEQ ID NO: 10 (Accession No. NP_{—}000624). In another embodiment, Bcl-X_{L} has the amino acid sequence as set forth in SEQ ID NO: 11 (Accession No. CAA80661). In another embodiment, Bcl-w has the amino acid sequence as set forth in SEQ ID NO: 12 (Accession No. AAB09055). In another embodiment, Bcl-B has the amino acid sequence as set forth in SEQ ID NO: 13 (Accession No. NP_{—}065129). In another embodiment, Mcl-1 has the amino acid sequence as set forth in SEQ ID NO: 14 (Accession No. AAF64255). In another embodiment, A1 (Bfl-1) has the amino acid sequence as set forth in SEQ ID NO: (Accession No. NP_{—}004040).

As used herein the term “apoptosis” or “apoptotic cell death” refers to programmed cell death which can be characterized by cell shrinkage, membrane blebbing and chromatin condensation culminating in cell fragmentation. Cells undergoing apoptosis also display a characteristic pattern of DNA cleavage. Alternatively, apoptosis can be characterized indirectly by changes in the activity or expression of members of the apoptotic pathway, e.g. activation of apoptotic caspases and mitochondrial release of cytochrome c.

Methods for determining cell apoptosis (or alternatively cell survival) are well known to a man skilled in the art. Apoptosis can be detected in populations of cells or in individual cells. Assays for determining cell apoptosis include but are not limited to:

TUNEL (TdT-mediated dUTP Nick-End Labeling) analysis which detects fragmented DNA, which occurs close to the final step in the apoptotic process. Fragmented DNA of apoptotic cells can incorporate fluorescein-dUTP at 3′-OH DNA ends using the enzyme Terminal Deoxynucleotidyl Transferase (TdT), which forms a polymeric tail using the principle of the TUNEL assay. The labeled DNA can then be visualized directly by fluorescence microscopy or quantitated by flow cytometry.

Annexin-V analysis that measures alterations in plasma membranes, detection of apoptosis related proteins such p53 and Fas. Annexin V is an anticoagulant protein that preferentially binds negatively charged phospholipids. An early step in the apoptotic process is disruption of membrane phospholipid asymmetry, exposuring phosphatidylserine (PS) on the outer leaflet of the plasma membrane. Fluorescently conjugated Annexin V can be used to detect this externalization of phosphatidylserine on intact living cells. Propidium iodide (PI) is often combined as a second fluorochrome to evaluate membrane integrity.

ISEL (in situ end labeling), and DNA laddering analysis for the detection of fragmentation of DNA in populations of cells or in individual cells.

Caspase activity assays-induction of apoptosis leads to procaspase-3 (32 kDa) proteolytic cleavage to generate an active 18 kDa caspase-3 fragment which then targets key modulators of the apoptotic pathway including poly-ADP-ribose polymerase and other caspases, for cleavage. Antibodies which recognize only the active 18 kDa fragment as a specific marker for apoptosis are commercially available.

The term “hyperproliferative disease” or “proliferative disease” as used herein, refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The term “neoplastic disease,” as used herein, refers to any abnormal growth of cells being either benign (non-cancerous) or malignant (cancerous).

The term “anti-neoplastic agent,” as used herein, refers to any compound that retards the proliferation, growth, or spread of a targeted (e.g., malignant) neoplasm.

The terms “sensitize” and “sensitizing,” as used herein, refer to making, through the administration of a first agent (e.g., the agent of the invention), an animal or a cell within an animal more susceptible, or more responsive, to the biological effects (e.g., promotion or retardation of an aspect of cellular function including, but not limited to, cell growth, proliferation, invasion, angiogenesis, or apoptosis) of a second agent (e.g., a chemotherapeutic agent). The sensitizing effect of a first agent on a target cell can be measured as the difference in the intended biological effect (e.g., promotion or retardation of an aspect of cellular function including, but not limited to, cell growth, proliferation, invasion, angiogenesis, or apoptosis) observed upon the administration of a second agent with and without administration of the first agent. The response of the sensitized cell can be increased by at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, or at least 500% over the response in the absence of the first agent.

Methods for determining the effect of an agent in enhancing apoptosis in cancer cells are well known in the art. Non limiting examples of cancer cells are HeLa cells, HEK293 cells and A549 cells as exemplified herein below.

The term “mediation of apoptosis” or “dysregulated apoptosis” as used herein, refers to any aberration in the ability of a cell to undergo cell death via apoptosis. Dysregulated apoptosis is associated with or induced by a variety of conditions, including for example, autoimmune disorders (e.g., systemic lupus erythematosus, rheumatoid arthritis, graft-versus-host disease, myasthenia gravis, or Sjogren's syndrome), chronic inflammatory conditions (e.g., psoriasis, asthma or Crohn's disease), hyperproliferative disorders (e.g., breast cancer, lung cancer), viral infections (e.g., herpes, papilloma, or HIV), and other conditions, such as osteoarthritis and atherosclerosis. It should be noted that when the deregulation is induced by or associated with a viral infection, the viral infection may or may not be detectable at the time deregulation occurs or is observed. That is, viral-induced deregulation can occur even after the disappearance of symptoms of viral infection.

The term “peptide” as used herein encompasses native peptides (degradation products, synthetic peptides or recombinant peptides), peptidomimetics (typically including non peptide bonds or other synthetic modifications) and the peptide analogues peptoids and semipeptoids, and may have, for example, modifications rendering the peptides more stable while in the body or more capable of penetrating into cells. Peptides typically consist of a sequence of about 3 to about 50 amino acids. According to a particular embodiment, the isolated peptides of the present invention consist of 5-50 amino acids, 5-45 amino acids, 5-40 amino acids, 5-35 amino acids, 5-30 amino acids, 5-28 amino acids, 5-26 amino acids, 5-24 amino acids, 5-22 amino acids, 5-20 amino acids, 6-18 amino acids, 5-16 amino acids, 5-14 amino acids, 5-12 amino acids or 5-10 amino acids, wherein each possibility represents a separate embodiment of the present invention.

The terms “polypeptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “isolated peptide” refers to a peptide that is essentially free from contaminating cellular components, such as carbohydrate, lipid, or other proteinaceous impurities associated with the peptide in nature. Typically, a preparation of isolated peptide contains the peptide in a highly purified form, i.e., at least about 80% pure, at least about 90% pure, at least about 95% pure, greater than 95% pure, or greater than 99% pure.

One of skill in the art will recognize that individual substitutions, deletions or additions to a peptide, or protein sequence which alters, adds or deletes a single amino acid or a small percentage of amino acids in the encoded sequence is a conservatively modified variant where the alteration results in the substitution of an amino acid with a similar charge, size, and/or hydrophobicity characteristics, such as, for example, substitution of a glutamic acid (E) to aspartic acid (D). Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W) (see, e.g., Creighton, Proteins, 1984).

The term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention.

The phrase “conservative substitution” also includes the use of a chemically derivatized residue in place of a non-derivatized residue provided that such peptide displays the requisite function of as specified herein.

The term “derived from” or “corresponding to” refers to construction of a peptide based on the knowledge of a sequence using any one of the suitable means known to one skilled in the art, e.g. chemical synthesis in accordance with standard protocols in the art. A peptide derived from, or corresponding to Atg12 BH3-like motif, or its proximal loop, can be an analog, fragment, conjugate (e.g. a lipopeptide conjugate) or derivative of a native Atg12 BH3-like motif, or its proximal loop, respectively, and salts thereof, as long as said peptide retains its ability to inhibit cell apoptosis.

Typically, the present invention encompasses derivatives of the peptides. The term “derivative” or “chemical derivative” includes any chemical derivative of the peptide having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine may be substituted for lysine.

In addition, a peptide derivative can differ from the natural sequence of the peptides of the invention by chemical modifications including, but are not limited to, terminal-NH₂ acylation, acetylation, or thioglycolic acid amidation, and by terminal-carboxlyamidation, e.g., with ammonia, methylamine, and the like. Peptides can be either linear, cyclic or branched and the like, which conformations can be achieved using methods well known in the art.

The peptide derivatives and analogs according to the principles of the present invention can also include side chain bond modifications, including but not limited to —CH₂—NH—, —CH₂—S—, —CH₂—S═O, O═C—NH—, —CH₂—O—, —CH₂—CH₂—, S═C—NH—, and —CH═CH—, and backbone modifications such as modified peptide bonds.

### Peptide Bonds

(—CO—NH—) within the peptide can be substituted, for example, by N-methylated bonds
(—N(CH3)-CO—); ester bonds (—C(R)H—C—O—O—C(R)H—N); ketomethylene bonds (—CO—CH2-); α-aza bonds (—NH—N(R)—CO—), wherein R is any alkyl group, e.g., methyl; carba bonds (—CH2-NH—); hydroxyethylene bonds (—CH(OH)—CH2-); thioamide bonds
(—CS—NH); olefinic double bonds (—CH═CH—); and peptide derivatives (—N(R)—CH2-CO—), wherein R is the “normal” side chain, naturally presented on the carbon atom. These modifications can occur at one or more of the bonds along the peptide chain and even at several (e.g., 2-3) at the same time.

The present invention also encompasses peptide derivatives and analogs in which free amino groups have been derivatized to form amine hydrochlorides, p-toluene sulfonylamino groups, carbobenzoxyamino groups, t-butyloxycarbonylamino groups, chloroacetylamino groups or formylamino groups. Free carboxyl groups may be derivatized to form, for example, salts, methyl and ethyl esters or other types of esters or hydrazides. The imidazole nitrogen of histidine can be derivatized to form N-im-benzylhistidine.

The peptide analogs can also contain non-natural amino acids. Examples of non-natural amino acids include, but are not limited to, sarcosine (Sar), norleucine, ornithine, citrulline, diaminobutyric acid, homoserine, isopropyl Lys, 3-(2′-naphtyl)-Ala, nicotinyl Lys, amino isobutyric acid, and 3-(3′-pyridyl-Ala).

Furthermore, the peptide analogs can contain other derivatized amino acid residues including, but not limited to, methylated amino acids, N-benzylated amino acids, O-benzylated amino acids, N-acetylated amino acids, O-acetylated amino acids, carbobenzoxy-substituted amino acids and the like. Specific examples include, but are not limited to, methyl-Ala (MeAla), MeTyr, MeArg, MeGlu, MeVal, MeHis, N-acetyl-Lys, O-acetyl-Lys, carbobenzoxy-Lys, Tyr-O-Benzyl, Glu-O-Benzyl, Benzyl-His, Arg-Tosyl, t-butylglycine, t-butylalanine, phenylglycine, cyclohexylalanine, and the like.

The peptides of the invention may be synthesized or prepared by techniques well known in the art. The peptides can be synthesized by a solid phase peptide synthesis method of Merrifield (see J. Am. Chem. Soc., 85:2149, 1964). Alternatively, the peptides of the present invention can be synthesized using standard solution methods well known in the art (see, for example, Bodanszky, M., Principles of Peptide Synthesis, Springer-Verlag, 1984) or by any other method known in the art for peptide synthesis.

In general, these methods comprise sequential addition of one or more amino acids or suitably protected amino acids to a growing peptide chain bound to a suitable resin.

Normally, either the amino or carboxyl group of the first amino acid is protected by a suitable protecting group. The protected or derivatized amino acid can then be either attached to an inert solid support (resin) or utilized in solution by adding the next amino acid in the sequence having the complimentary (amino or carboxyl) group suitably protected, under conditions conductive for forming the amide linkage. The protecting group is then removed from this newly added amino acid residue and the next amino acid (suitably protected) is added, and so forth. After all the desired amino acids have been linked in the proper sequence, any remaining protecting groups are removed sequentially or concurrently, and the peptide chain, if synthesized by the solid phase method, is cleaved from the solid support to afford the final peptide.

In the solid phase peptide synthesis method, the alpha-amino group of the amino acid is protected by an acid or base sensitive group. Such protecting groups should have the properties of being stable to the conditions of peptide linkage formation, while being readily removable without destruction of the growing peptide chain. Suitable protecting groups are t-butyloxycarbonyl (BOC), benzyloxycarbonyl (Cbz), biphenylisopropyloxycarbonyl, t-amyloxycarbonyl, isobornyloxycarbonyl, (alpha,alpha)-dimethyl-3,5-dimethoxybenzyloxycarbonyl, o-nitrophenylsulfenyl, 2-cyano-t-butyloxycarbonyl, 9-fluorenylmethyloxycarbonyl (FMOC) and the like. The BOC or FMOC protecting group is preferred.

In the solid phase peptide synthesis method, the C-terminal amino acid is attached to a suitable solid support. Suitable solid supports useful for the above synthesis are those materials, which are inert to the reagents and reaction conditions of the stepwise condensation-deprotection reactions, as well as being insoluble in the solvent media used. Suitable solid supports are chloromethyl polystyrene-divinylbenzene polymer, hydroxymethyl-polystyrene-divinylbenzene polymer, and the like. The coupling reaction is accomplished in a solvent such as ethanol, acetonitrile, N,N-dimethylformamide (DMF), and the like. The coupling of successive protected amino acids can be carried out in an automatic polypeptide synthesizer as is well known in the art.

The peptides of the invention may alternatively be synthesized such that one or more of the bonds, which link the amino acid residues of the peptides are non-peptide bonds. These alternative non-peptide bonds include, but are not limited to, imino, ester, hydrazide, semicarbazide, and azo bonds, which can be formed by reactions well known to skilled in the art.

The peptides of the present invention, analogs, or derivatives thereof produced by recombinant techniques can be purified so that the peptides will be substantially pure when administered to a subject. The term “substantially pure” refers to a compound, e.g., a peptide, which has been separated from components, which naturally accompany it. Typically, a peptide is substantially pure when at least 50%, preferably at least 75%, more preferably at least 90%, and most preferably at least 99% of the total material (by volume, by wet or dry weight, or by mole percent or mole fraction) in a sample is the peptide of interest. Purity can be measured by any appropriate method, e.g., in the case of peptides by HPLC analysis.

Included within the scope of the invention are peptide conjugates comprising the peptides of the present invention derivatives, or analogs thereof joined at their amino or carboxy-terminus or at one of the side chains via a peptide bond to an amino acid sequence of a different protein. Conjugates comprising peptides of the invention and a protein can be made by protein synthesis, e.g., by use of a peptide synthesizer, or by ligating the appropriate nucleic acid sequences encoding the desired amino acid sequences to each other by methods known in the art, in the proper coding frame, and expressing the conjugate by methods commonly known in the art. Additionally or alternatively, the peptides of the present invention, derivatives, or analogs thereof can be joined to another moiety such as, for example, a fatty acid, a sugar moiety, arginine residues, hydrophobic moieties, and any known moiety that facilitate membrane or cell penetration.

Addition of amino acid residues may be performed at either terminus of the peptides of the invention for the purpose of providing a “linker” by which the peptides of this invention can be conveniently bound to a carrier. Such linkers are usually of at least one amino acid residue and can be of 40 or more residues, more often of 1 to 10 residues. Typical amino acid residues used for linking are tyrosine, cysteine, lysine, glutamic and aspartic acid, or the like.

Pharmaceutical Compositions

According to some embodiments, the present invention provides a pharmaceutical composition comprising as an active ingredient an agent capable of mediating Atg12 binding to a Bcl-2 member, and a pharmaceutically acceptable carrier, excipient or diluent.

As used herein, a “pharmaceutical composition” refers to a preparation of one or more of the active ingredients described herein, with other components such as physiologically suitable carriers and excipients. The purpose of a pharmaceutical composition is to facilitate administration of a compound to a subject.

Hereinafter, the phrases “therapeutically acceptable carrier” and “pharmaceutically acceptable carrier”, which may be used interchangeably, refer to a carrier or a diluent that does not cause significant irritation to an organism and does not abrogate the biological activity and properties of the administered compound.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

In another embodiment of the present invention, a therapeutic composition further comprises a pharmaceutically acceptable carrier. As used herein, a “carrier” refers to any substance suitable as a vehicle for delivering of the agents or molecule of the present invention to a suitable in vivo or in vitro site. As such, carriers can act as a pharmaceutically acceptable excipient of a therapeutic composition of the present invention. Carriers of the present invention include: (1) excipients or formularies that transport, but do not specifically target a molecule to a cell (referred to herein as non-targeting carriers); and (2) excipients or formularies that deliver a molecule to a specific site in a subject or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Therapeutic compositions of the present invention can be sterilized by conventional methods.

Targeting carriers are herein referred to as “delivery vehicles”. Delivery vehicles of the present invention are capable of delivering a therapeutic composition of the present invention to a target site in a subject. A “target site” refers to a site in a subject to which one desires to deliver a therapeutic composition. Examples of delivery vehicles include, but are not limited to, artificial and natural lipid-containing delivery vehicles. Natural lipid-containing delivery vehicles include cells and cellular membranes. Artificial lipid-containing delivery vehicles include liposomes and micelles. A delivery vehicle of the present invention can be modified to target to a particular site in a subject, thereby targeting and making use of a nucleic acid molecule of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. For example, an antibody specific for an antigen found on the surface of a target cell can be introduced to the outer surface of a liposome delivery vehicle so as to target the delivery vehicle to the target cell. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

Therapeutic Use

According to some embodiments, the peptides or agents of the present invention are useful in regulating Atg12 apoptotic activity (e.g., by reducing or enhancing Atg12 binding to Bcl-2 anti-apoptotic family members). Promoting Atg12 apoptotic activity is beneficial in the treatment of hyper proliferative diseases such as cancer. Reducing Atg12 apoptotic activity is beneficial in the treatment of diseases characterized by excessive cell death such as neurodegenerative diseases.

According to other embodiments, the present invention provides a method for treating a disease or disorder characterized by excessive cellular death in a subject in need thereof, comprising administering to the subject a therapeutically effective amount of a pharmaceutical composition comprising the anti-apoptotic agent obtained by the anti-apoptotic screening method of the invention.

In some embodiments the disease or disorder characterized by excessive cellular death is selected from immunodeficiency diseases (e.g., AIDS), senescence, neurodegenerative diseases, ischemia and reperfusion, infertility, wound-healing, stroke, myocardial infarction, cardiac disease, hypertension, septic shock, organ transplantation and ophthalmic diseases (e.g., diabetic retinopathy and age-related macular degeneration (AMD)).

According to some embodiments, the present invention is directed to a method for treating cancer in a subject in need thereof, comprising administering to the subject a pharmaceutical composition comprising a therapeutically effective amount of an agent capable of promoting Atg12 apoptotic activity or alternatively, mimicking Atg12's apoptotic activity by inhibiting Bcl-2 anti-apoptotic members. According to certain embodiments, the cancer is associated with Bcl-2 overexpression.

In another embodiment, the invention provides a method for inhibiting tumor progression in a subject in need thereof, comprising administering to the subject a pharmaceutical composition comprising a therapeutically effective amount of an agent capable of promoting Atg12 apoptotic activity.

In another embodiment, there is provided a method for inducing tumor regression in a subject in need thereof, comprising administering to the subject a pharmaceutical composition comprising a therapeutically effective amount of an agent capable of promoting Atg12 apoptotic activity.

The compounds of the present invention are active against a wide range of cancers, including carcinomas, sarcomas, myelomas, leukemias, lymphomas and mixed type tumors. Particular categories of tumors amenable to treatment include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors amenable to treatment include: hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

According to certain embodiments, the cancer to be treated is selected from the group consisting of prostate cancer, breast cancer, skin cancer, colon cancer, lung cancer, pancreatic cancer, lymphoma, myeloma, leukemia, head and neck cancer, kidney cancer, ovarian cancer, bone cancer, liver cancer or thyroid cancer.

For example, in some embodiments, the tumor may include pediatric solid tumors, e.g., Wilms' tumor, hepatoblastoma and embryonal rhabdomyosarcoma, wherein each possibility represents a separate embodiment of the present invention. In other embodiments, the tumor includes, but is not limited to, germ cell tumors and trophoblastic tumors (e.g. testicular germ cell tumors, immature teratoma of the ovary, sacrococcygeal tumors, choriocarcinoma and placental site trophoblastic tumors), wherein each possibility represents a separate embodiment of the present invention. According to additional embodiments, the tumor includes, but is not limited to, epithelial adult tumors (e.g. bladder carcinoma, hepatocellular carcinoma, ovarian carcinoma, cervical carcinoma, lung carcinoma, breast carcinoma, squamous cell carcinoma in head and neck, colon carcinoma, renal cell carcinoma and esophageal carcinoma), wherein each possibility represents a separate embodiment of the present invention. In yet further embodiments, the tumor includes, but is not limited to, neurogenic tumors (e.g. astrocytoma, ganglioblastoma and neuroblastoma), wherein each possibility represents a separate embodiment of the present invention. In another embodiment, the tumor is prostate cancer. In another embodiment, the tumor is pancreatic cancer. In other embodiments, the tumor includes, for example, Ewing sarcoma, congenital mesoblastic nephroma, gastric adenocarcinoma, parotid gland adenoid cystic carcinoma, duodenal adenocarcinoma, T-cell leukemia and lymphoma, nasopharyngeal angiofibroma, melanoma, osteosarcoma, uterus cancer and non-small cell lung carcinoma, wherein each possibility represents a separate embodiment of the present invention.

Combination Therapy with Chemotherapy

In certain embodiments, the agents and/or peptides of the present invention can be used to treat cancer alone or in combination with other established or experimental therapeutic regimens against cancer, which can act in an additive or synergistic manner with it. Therapeutic methods for treatment of cancer suitable for combination with the present invention include, but are not limited to, chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy, and photon beam radiosurgical therapy.

According to certain aspects, administration of the apoptotic agent of the invention in conjunction with at least one antitumor chemotherapeutic agent acts to enhance the antitumor effect of chemotherapeutic agents. In preferred embodiments, the combinations of the apoptotic agent of the invention together with the at least one chemotherapeutic agent improve the clinical outcome in a significant manner versus each of the treatments alone. In a preferred embodiment, there is synergy when tumors are treated with the apoptotic agent of the invention in conjunction with at least one chemotherapeutic agent, and, optionally further in conjunction with radiation.

Antitumor effect induced by the combinations of the invention includes the prevention, inhibition of the progression of a tumor, reduction of tumor growth and protection against tumor recurrence, including cancerous and noncancerous tumors. The progression of a tumor includes the invasiveness, metastasis, recurrence and increase in size of the tumor. The reduction of tumor growth also includes the destruction or elimination of a tumor leading to complete remission.

The invention further provides a method of enhancing survival in a subject with a tumor, which comprises administration of the apoptotic agent of the invention, either on its own, or optionally, combined with the further administration of one or more chemotherapeutic agents.

The invention further provides a method of reducing or preventing recurrence of a tumor, which comprises administration of the apoptotic agent of the invention, either on its own, or optionally, combined with the further administration of one or more chemotherapeutic agents.

According to another embodiment, the present invention provides a method of treating a tumor, the method comprising (i) administering to a subject in need thereof an effective amount of an apoptotic agent of the invention; and (ii) administering to the subject an effective amount of at least one chemotherapeutic agent; thereby treating the tumor.

According to yet another embodiment of the invention there is provided a method of enhancing survival or inhibiting disease progression in a subject having a tumor, wherein the subject is treated with at least one chemotherapeutic agent, the method comprising administering an effective amount of an apoptotic agent of the invention, thereby enhancing survival of the subject.

According to yet another embodiment, the invention provides a method of reducing or preventing tumor recurrence, the method comprising administering to a subject in need thereof an effective amount of an apoptotic agent of the invention, thereby reducing or preventing tumor recurrence. According to one embodiment, the method of reducing or preventing tumor recurrence further comprises administering to the subject at least one chemotherapeutic agent. According to particular embodiments, the subject is undergoing or has completed a course of chemotherapy with at least one chemotherapeutic agent.

According to various embodiments, the administering of the apoptotic agent of the invention and of the at least one chemotherapeutic agent is carried out substantially simultaneously, concurrently, alternately, sequentially or successively. In some embodiments, said agent and the at least one chemotherapeutic agent are administered according to overlapping schedules.

According to particular embodiments, administering of the apoptotic agent of the invention is carried out prior to initial administration of the at least one chemotherapeutic agent.

According to other embodiments, administering of either or both of the apoptotic agent of the invention and the at least one chemotherapeutic agent is carried out by a route selected from the group consisting of intravenous, oral, intraperitoneal, subcutaneous, isolated limb perfusion, infusion into an organ and combinations thereof.

In particular embodiments, the methods of the invention further comprise assessing at least one parameter selected from the group consisting of: rate of tumor growth, tumor volume, number of metastases, tumor recurrence and combinations thereof.

It should be noted that according to the teaching of the present invention, the apoptotic agent of the invention may be administered before, during, or after commencing chemotherapy and, optionally, radiation therapy, as well as any combination thereof, i.e. before and during, before and after, during and after, or before, during, and after commencing the chemotherapy and, optionally, the radiation therapy. For example, the agent of the invention of the invention may be administered between 1 and 30 days prior to or after commencing chemotherapy. The agent may further be administered between courses of chemotherapy.

In the combination therapy methods of the invention, the apoptotic agent of the invention may be administered in parallel to the chemotherapy, for example substantially simultaneously or concurrently. Other administration schedules may also be used, for example, overlapping schedules or those which involve alternately, sequentially or successively administering the two types of treatment.

According to various embodiments, the at least one chemotherapeutic agent is selected from the group consisting of: antimetabolites, platinum-based drugs, mitotic inhibitors, anthracycline antibiotics, topoisomerase inhibitors, anti-angiogenic agents and combinations thereof.

Accordingly, in various embodiments, the chemotherapeutic agent may be selected from an antimetabolite, such as the pyrimidine analog 5-fluorouracil, or cytarabin, or a platinum-based drug, such as oxaliplatin or cisplatin. Further, in various embodiments, the chemotherapeutic agent may be other than an agent selected from a topoisomerase I inhibitor (such as SN-38) and an alkylating agent (such as cyclophosphamide).

According to some embodiments, the at least one chemotherapeutic agent is an antimetabolite, including purine antagonists, pyrimidine antagonists and folate antagonists. According to some embodiments, the antimetabolite is a pyrimidine antagonist. According to some embodiments, the antimetabolite is selected from the group consisting of: 5-fluorouracil, uracil mustard, uracil, capecitabine, 6-mercaptopurine, methotrexate, gemcitabine, cytarabine, fludarabine, and pemetrexed.

According to some embodiments, the at least one chemotherapeutic agent is 5-fluorouracil. According to some embodiments, the at least one chemotherapeutic agent is cytarabine. According to some embodiments, the at least one chemotherapeutic agent is a platinum-based drug selected from the group consisting of: cisplatin, carboplatin and oxaliplatirn. According to yet other embodiments, the at least one chemotherapeutic agent is a mitotic inhibitor selected from the group consisting of: paclitaxel, docetaxel, etoposide, vinblastine, vincristine and vinorelbine. According to yet other embodiments, the at least one chemotherapeutic agent is an anthracycline antibiotic selected from the group consisting of: daunorubicin, respinomycin D and idarubicin. According to some embodiments, the at least one chemotherapeutic agent is an anti-angiogenic agent selected from the group consisting of: bevacizumab, dopamine, tetrathiomolybdate, and antiangiogenic variants of VEGF.

Chemotherapy drugs are divided into several groups based on their effect on cancer cells, the cellular activities or processes the drug interferes with, or the specific phases of the cell cycle the drug affects. Accordingly, chemotherapy drugs fall in one of the following categories: alkylating agents, nitrosoureas, antimetabolites, anthracyclines, topoisomerase I and II inhibitors, mitotic inhibitors, inter alia platinum based drugs, steroids and anti-angiogenic agents.

Antimetabolites, also termed “nucleoside analogs”, replace natural substances as building blocks in DNA molecules, thereby altering the function of enzymes required for cell metabolism and protein synthesis. In the event that they mimic nutrients required for cell growth, the cells eventually undergo lysis. If a nucleoside is replaced with a non-functional nucleoside analog, the latter is incorporated into DNA and RNA, finally inducing cell cycle arrest and apoptosis by inhibiting the cell's ability to synthesize DNA. Antimetabolites are cell-cycle specific and are most effective during the S-phase of cell division as they primarily act upon cells undergoing synthesis of new DNA for formation of new cells. The toxicities associated with these drugs are seen in cells that are growing and dividing quickly. Examples of antimetabolites include purine antagonists, pyrimidine antagonists, and folate antagonists. These agents damage cells during the S phase and are commonly used to treat leukemias, tumors of the breast, ovary, and the gastrointestinal tract, as well as other cancers. Specific examples of antimetabolites include 5-fluorouracil (also known as 5FU), capecitabine, 6-mercaptopurine, methotrexate, gemcitabine, cytarabine, fludarabine and pemetrexed.

Platinum-based chemotherapeutic drugs crosslink DNA in several different ways, interfering with cell division by mitosis. The damaged DNA elicits DNA repair mechanisms, which in turn activate apoptosis when repair proves impossible. Most notable among the DNA changes are the 1,2-intrastrand cross-links with purine bases. These include 1,2-intrastrand d(GpG) adducts which form nearly 90% of the adducts and the less common 1,2-intrastrand d(ApG) adducts. 1,3-intrastrand d(GpXpG) adducts occur but are readily excised by the nucleotide excision repair (NER). Other adducts include inter-strand crosslinks and nonfunctional adducts that have been postulated to contribute to the activity of platinum-based drugs. Interaction with cellular proteins, particularly HMG domain proteins, has also been advanced as a mechanism of interfering with mitosis, although this is probably not its primary method of action. Platinum-based chemotherapeutic drugs include cisplatin (also known as cisplatinum or cis-diamminedichloridoplatinum II (CDDP), carboplatin and oxaliplatin. Cisplatin is frequently designated as an alkylating agent, though it has no alkyl group and cannot carry out alkylating reactions. It is correctly classified as alkylating-like. Platinum-based chemotherapeutic drugs are used to treat various types of cancers, including sarcomas, some carcinomas (e.g. small cell lung cancer, and ovarian cancer), lymphomas and germ cell tumors.

Mitotic inhibitors interfere with cell division. The most known chemotherapeutic agent in this category is paclitaxel (also known as Taxol®, “plant alkaloid”, “taxane” and an “antimicrotubule agent”). Together with docetaxel, it forms the drug category of the taxanes. However, other mitotic inhibitors are known, including, but not limited to etoposide, vinblastine and vincristine. Paclitaxel acts by interfering with normal microtubule growth during cell division by arrests their function; it hyper-stabilizes their structure. This destroys the cell's ability to use its cytoskeleton in a flexible manner. Specifically, paclitaxel binds to the β subunit of tubulin, the “building block” of microtubules, and the binding of paclitaxel locks these building blocks in place. The resulting microtubule/paclitaxel complex does not have the ability to disassemble. This adversely affects cell function because the shortening and lengthening of microtubules (termed dynamic instability) is necessary for their function as a mechanism to transport other cellular components. For example, during mitosis, microtubules position the chromosomes all through their replication and subsequent separation into the two daughter-cell nuclei. Furthermore, paclitaxel induces programmed cell death (apoptosis) in cancer cells by binding to the apoptosis stopping protein Bcl-2 (B-cell leukemia 2) and thus arresting its function.

Another group of DNA-interacting drugs widely used in anti-cancer chemotherapy is the group of anthracycline antibiotics which includes, inter alia, daunorubicin, doxorubicin (also known as Adriamycin® and doxorubicin hydrochloride), respinomycin D and idarubicin. These drugs interact with DNA by intercalation and inhibition of macromolecular biosynthesis thereby inhibiting the progression of the enzyme topoisomerase II, which unwinds DNA for transcription. They stabilize the topoisomerase II complex after it has broken the DNA chain for replication, preventing the DNA double helix from being resealed and thereby stopping the process of replication. It is commonly used in the treatment of a wide range of cancers.

Alkylating antineoplastic agents directly attack DNA. They attach an alkyl group to DNA, cross-linking guanine nucleobases in DNA double-helix strands. This makes the strands unable to uncoil and separate. As this is necessary in DNA replication, the cells can no longer divide. These drugs act nonspecifically. Cyclophosphamide is an alkylating agent, however, it is a highly potent immunosuppressive substance.

Topoisomerase I and II inhibitors interfere with the enzymatic activity of topoisomerase I and 2, respectively, eventually leading to inhibition of both DNA replication and transcription. Examples of topoisomerase I inhibitors include topotecan and irinotecan. Irinotecan, is a prodrug converted to a biologically active metabolite 7-ethyl-10-hydroxy-camptothecin (SN-38) by a carboxylesterase-converting enzyme. One thousand-fold more potent than its parent compound irinotecan, SN-38 inhibits topoisomerase I activity by stabilizing the cleavable complex between topoisomerase I and DNA, resulting in DNA breaks that inhibit DNA replication and trigger apoptotic cell death. Because ongoing DNA synthesis is necessary for irinotecan to exert its cytotoxic effects, it is also classified as an S-phase-specific agent. Examples of topoisomerase II inhibitors include etoposide and teniposide.

Anti-angiogenic agents interfere with the generation of new blood vessels, eventually leading to the “starvation” of tumors. Non-limiting examples of anti-angiogenic agents include the monoclonal antibody bevacizumab, dopamine and tetrathiomolybdate.

Vascular endothelial growth factor (VEGF) is a 32-42 kDa dimeric glycoprotein which mediates vasodilatation, increased vascular permeability and endothelial cell mitogenesis. Differential exon splicing of the VEGF gene results in three main mRNA species which code for three secreted isoforms (subscripts denote numbers of amino acids): VEGF189, VEGF165, and VEGF121. A number of minor splice variants have also been described (VEGF206, VEGF183, VEGF145 and VEGF148). Variants of VEGF polypeptides and their use in cancer therapy are disclosed for example, in WO/2003/012105.

Screening Assays

The present invention provides screening assays for identifying agents that modulate (e.g., disrupt or enhance) the binding of atg12 to Bcl-2 family members (e.g., anti-apoptotic Bcl-2 members), thereby regulating Atg12 apoptotic activity.

In one embodiment, the method of screening described herein is a screening assay, such as a high-throughput screening assay. Thus, the contacting step can be in a cell-based or cell-free assay. For example, cells expressing Atg12 and a Bcl-2 anti-apoptotic member can be contacted with a candidate agent either in vivo, ex vivo, or in vitro. The cells can be assayed in vitro or in situ or the protein extract of said cells can be assayed in vitro for the detection of Atg12 binding to a Bcl-2 anti-apoptotic member. Cells can also be engineered to express a reporter construct, wherein the cells are contacted with a candidate agent and evaluated for reporter expression. Other such cell-based and cell-free assays are contemplated for use herein.

For example, the effect of small molecule, amino acid or nucleic acid mimetics on cancer cells or other pathologic associated with dysregulated apoptosis can be evaluated in cells expressing Atg12 and compared to cells lacking Atg12.

In general, candidate (potential) agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein.

A potential agent may be a single compound of interest or a member of a library of potential inhibitors. For example, a library of potential agents or molecules may be a synthetic combinatorial library (e.g., a combinatorial chemical library), a cellular extract, a bodily fluid (e.g., urine, blood, tears, sweat, or saliva), or other mixture of synthetic or natural products (e.g., a library of small molecules or a fermentation mixture). A library of potential agents can include, for example, amino acids, peptides, polypeptides, proteins (including, but not limited to, antibodies, antibody fragments and peptide aptamers), or fragments of peptides or proteins; nucleic acids (e.g., DNA; RNA; or peptide nucleic acids, PNA); aptamers; or compounds such as carbohydrates and polysaccharides. Each member of the library can be singular or can be a part of a mixture (e.g., a compressed library). The library can contain purified compounds or can be “dirty” (i.e., containing a significant quantity of impurities). Commercially available libraries (e.g., from Affymetrix, ArQule, Neose Technologies, Sarco, Ciddco, Oxford Asymmetry, Maybridge, Aldrich, Panlabs, Pharmacopoeia, Sigma, or Tripose) can also be used with the new methods.

Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect should be employed whenever possible.

In addition to libraries of potential agents, special libraries called diversity files can be used to assess the specificity, reliability, or reproducibility of the new methods. Diversity files contain a large number of compounds (e.g., 1000 or more small molecules) representative of many classes of compounds that could potentially result in nonspecific detection in an assay. Diversity files are commercially available or can also be assembled from individual compounds commercially available from the vendors listed above.

Peptide aptamers represent a novel generation of molecules in which variable peptides are inserted into a protein scaffold. As such, they can bind to their target in vivo and have the potential to selectively block its activity. Several bacterial proteins had been recently applied as scaffolds for peptide aptamers, including thioredoxin, staphylococcal nuclease and alpha-amylase, as well as non-bacterial proteins such as green fluorescent protein (Colas, 2000) (Hoppe-Seyler and Butz, 2000). The scaffold share intrinsic stability making it possible to express peptide aptamers in vivo at high concentrations, and, having the peptide aptamer been identified, utilize its high level expression and easy purification for subsequent analysis. Once identified, a peptide aptamer may be evaluated as a free peptide, where in some cases it is as active as in the context of the aptamer (Hoppe-Seyler and Butz, 2000). Moreover, small synthetic molecules may be derived from such bioactive aptamers to form the basis of new therapeutics.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits vascular permeability. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions in which it is desirable to regulate vascular permeability.

The potential agent may include, but are not limited to, peptides or proteins (either recombinant or naturally occurring), nucleic acids or other organic or inorganic compounds (e.g. carbohydrates and polysaccharides). In one embodiment, the potential inhibitors are peptide aptamers, comprising a peptide fused to a stabilizing protein. In another embodiment, the potential inhibitors are antibodies, antibody fragments including, but not limited to, single-chain antibodies (scFvs) and single antibody domain proteins (dAbs).

Having now generally described the invention, the same will be more readily understood through reference to the following examples, which are provided by way of illustration and are not intended to be limiting of the present invention.

### EXAMPLES

### Materials and Methods

Cell Culture and Transfections

HEK293 and HeLa Cells were maintained in Dulbecco's Modified Eagle's Medium (DMEM; Biological Industries) supplemented with 2 mM glutamine (Gibco BRL), 100 U/ml penicillin and streptomycin (Gibco BRL), and 10% fetal bovine serum (Hyclone). ACHN cells were maintained in RPMI-1640 medium (Invitrogen). For transient transfection of DNA, cells were grown to approximately 60% confluence and transfected by the calcium phosphate method with the indicated plasmids. In the Mcl-1 inhibition experiment, transfection of DNA was performed using Lipofectamine-2000 reagent (invitrogen).

siRNA Screening and Caspase Activity Assay

HEK293 cells were reverse-transfected in 96-well plates with 50 nM of the indicated siGENOME siRNA pools (Dharmacon) using DharmaFECT4 transfection reagent (Dharmacon). 24 h post-transfection, cells were treated with 50 μM etoposide (Sigma Aldrich) for a period of 48 h. Caspase activity was measured using the Caspase-Glo 3/7 luminescent assay (Promega), according to manufacturer's instructions. Plates were read using the Veritas microplate luminometer (Turner BioSystems). siRNA targeting caspase-3 was used as positive control, while non-targeting pool, RISC-free siRNA and mock transfection were used as negative controls. The average caspase activity of negative controls was defined as 100% caspase activity. A similar protocol was used for hit validation using ON-TARGETpIus siRNA pools (Dharmacon) and individual siRNA duplexes (Sigma Aldrich). In the latter experiment, Sigma universal siRNA control #1 was used as an additional negative control. For cell number determination, DNA content was measured in parallel plates using the CyQUANT NF cell proliferation assay (Invitrogen)

Data from three biological repeats of the primary screen were normalized in an experiment-wise manner according to the sample median of each screen. All siRNAs were screened in three replicate wells per plate. Positive hits were defined as genes whose knockdown led to a statistically significant (p<0.01) decrease in caspase activity compared to the group of non-targeting controls in a student's two-tailed t-test in all three biological repeats of the screen.

Immunoblotting

Cell pellets were lysed in PLB buffer (100 mM NaCl, 10 mM NaPO₄, 5 mM EDTA pH 8.0, 1% Triton X-100, 0.5% sodium deoxycholate, 0.1% SDS) and subjected to western blot analysis as previously described (Bialik et al., 2008). The following antibodies were used: monoclonal antibodies to tubulin, actin, FLAG (Sigma Aldrich), PARP-1 (BioMol), CD95 (CH-11), Atg12 (MBL), cytochrome c, p62 (BD Pharmingen), Bcl-2 and Mcl-1 (Santa Cruz Biotechnology); polyclonal antibodies to ICAD, Atg12, Mcl-1, cleaved caspase-3, Bad (Cell Signaling), Atg5 and Atg3 (Sigma Aldrich). Detection was done with HRP-conjugated goat anti-mouse or anti-rabbit secondary antibodies (Jackson Immuno Research), followed by enhanced chemiluminescence (SuperSignal, Pierce). Densitometric analysis was performed using EZQuant-Gel software (EZQuant).

Cell Viability

Cell viability was assessed using the XTT colorimetric assay according to manufacturer's instructions (Biological industries). Absorbance was read in a microplate ELISA reader (Bio-Tek) at 450 nm. Membrane integrity was evaluated by propidium iodide (PI) uptake; PI (25 μg/ml; Sigma Aldrich) was added to cells immediately prior to analysis by flow cytometry (FACScan; Beckton Dickinson).

Clonogenic Survival

siRNA-transfected HeLa cells were treated with 0.5 M STS for 15 h, washed, and re-plated onto 6-well plates. Cells were allowed to grow for 5 days, fixed in 3.7% formaldehyde and stained with 0.05% (w/v) crystal violet (Sigma Aldrich). Surviving cells were imaged using a binocular microscope (Leica MZ16F).

Immunoprecipitation (IP)

For co-immunoprecipitation, HEK293 cells were transiently transfected with the indicated plasmids. Cell pellets were lysed in B buffer (0.4% NP-40, 0.5 mM EDTA, 100 mM KCl, 20 mM Hepes pH 7.6, 20% glycerol) supplemented with protease and phosphatase inhibitors. Following pre-clearance with protein G PLUS-Agarose beads (Santa Cruz Biotechnology), extracts were incubated with anti-FLAG M2 beads (Sigma Aldrich) for 2 h, eluted with excess FLAG peptide (Sigma Aldrich) and subjected to western blot analysis. In Example 7, an irrelevant tagged protein (DAP1-Flag) was used as control.

For endogenous IPs, mouse monoclonal antibody against Atg12 (MBL) was used for immunoprecipitation of Atg12. Detection of Mcl-1 was carried out with mouse anti-Mcl-1 (RC13, Santa Cruz Biotechnology), followed by incubation with rat anti-mouse Ig, kappa light chain secondary antibody (BD Pharmingen) to avoid detection of Ab heavy chain.

Conformationally active Bax was immunoprecipitated with anti-Bax antibody (6A7, Abcam). Cell pellets from STS-treated HeLa cells (3 μM, 4 h; Sigma Aldrich) were resuspended in 1% CHAPS (3-[(3-cholamidopropyl)-dimethylammonio]-1-propanesulfonate) buffer supplemented with protease and phosphatase inhibitors. Pre-cleared cell lysates were incubated with 2 μg antibody overnight at 4° C., followed by incubation with protein G agarose beads. Beads were washed with lysis buffer and boiled in sample buffer. Western blot detection of total Bax was performed with anti-Bax N20 antibody (Santa Cruz Biotechnology).

Multiple Sequence Alignment

Multiple sequence alignment was performed with the ClustalW algorithm (Thompson et al., 1994) and displayed using GeneDoc (http://www.psc.edu/biomed/genedoc).

Cell Fractionation

Cell pellets were resuspended in isotonic HIM buffer (200 mM mannitol, 70 mM sucrose, 1 mM EGTA, 10 mM HEPES, pH 7.5) with protease inhibitors, homogenized by multiple passages through a 25 G needle, and centrifuged at 550×g at 4° C. for 10 min. The supernatant (S1) was re-centrifuged at 10,000×g at 4° C. for 20 min to pellet mitochondria, and the supernatant was saved as the cytosolic fraction.

GFP-LC3 Punctate Staining

HEK293 cells stably expressing GFP-LC3 were plated on 13 mm glass coverslips coated with poly-L-Lysine (Sigma Aldrich), and subjected to starvation for 4 h in Earl's Balanced Salt Solution (EBSS; Biological industries). Cells were fixed in 3.7% formaldehyde and viewed by fluorescent microscopy (Olympus BX41) with 60× (N.A. 1.25) UPlan-Fl oil immersion objectives. Digital images were obtained with a DP50 CCD camera using ViewfinderLite and StudioLite software (Olympus). The percentage of puncta area per total cell area was determined using MetaMorph imaging software (Molecular Devices).

DNA Expression Vectors

Atg12 and Mcl-1 cDNAs were obtained from imaGenes, and sub-cloned into the pcDNA3 expression vector (invitrogen). Point mutations were generated using sitedirected mutagenesis (Agilent). siRNA-resistant constructs of Atg12 (Flag-tagged and untagged) were generated by introducing eight silent mutations to the siRNA binding site (GCA/C GTA/G GAG/A C/AGA/G ACA/C C/AGA/G). Bcl-2 plasmid was obtained from OriGene. Bax was a kind gift from A. Gross. GFP-LC3 was a kind gift from N. Mizushima and T. Yoshimori. Atg5-HA was a kind gift from Y. K. Jung.

Homology Modeling and Protein-Protein Docking

An initial model of Atg12 was constructed using the Homology module of InsightII (Accelrys Inc., San Diego, Calif.), based on the available plant Atg12 structure (Suzuki et al., 2005). The initial model was immersed in a cube of water and energy minimized using the Gromacs package (Lindhal, 2001). A short molecular dynamics (MD) simulation of 2 ns was conducted in order to obtain a reliable model for loop 93-97. The non-hydrogen atoms were restrained in the simulation except those in loops 93-97 and 105-108. Analysis of the simulation trajectory showed that the structure of loop 93-97 is stable in the last 1 ns. The central conformer was used in docking to Mcl-1. Two X-ray structures of Mcl-1 are available, one with bound ligand and significantly higher resolution (1.55 Å versus 2.80 Å). This structure (PDB code 2nl9) lacks a segment, which was completed based on the lower resolution structure (PDB code 2nla). This segment is far from the BH3 binding cavity and unlikely to be involved in Atg12 binding. Protein-protein docking was executed with the geometric-electrostatic-hydrophobic (GEH) version of MolFit (Berchanski et al., 2004). MolFit performs an exhaustive stepwise scan in the rotation/translation space; standard grid intervals were used (Kowalsman and Eisenstein, 2007). The search was not biased by biological information and produced 10,686 docking models assessed by the complementarity of the shape, electrostatic potential and hydrophobicity (GEH score). These models were re-evaluated with a post-screen filter that tests the residue propensity, pairwise propensity and interface desolvation energy and retains only models that pass the pre-calibrated thresholds (Kowalsman and Eisenstein, 2009). The 610 models that passed the filter were screened based on biological information, requiring that Atg12/D64 binds with any residue in the BH3 binding cavity of Mcl-1, retaining 14 models.

In-Vitro Kinase Assay

Recombinant Atg12 was expressed in E. coli and purified on FLAG-agarose beads (Sigma aldrich). To activate p38, p38 and MKK6 were incubated for 30 min at 30c in a kinase reaction buffer. Subsequently, activated p38 was incubated with Atg12 in the presence of 33P-ATP for 30 min at 30c. The reaction was stopped by boiling and the proteins were separated on acrylamide gel.

In-Gel Proteolysis and Mass Spectrometry Analysis

Gel bands were excised and subjected to mass spectrometric analysis at the Smoler Proteomics Center at the Technion (Haifa, Israel). The proteins in the gel were reduced with 10 mM DTT, modified with 40 mM iodoacetamide and trypsinized (modified trypsin (Promega)) at a 1:100 enzyme-to-substrate ratio. The resulting tryptic peptides were resolved by reverse-phase chromatography on 0.1×200-mm fused silica capillaries (J&W, 100 micrometer ID) packed with Everest reversed phase material (Grace Vydac, Calif.). The peptides were eluted with linear 120 minute gradients of 5 to 95% of acetonitrile with 0.1% formic acid in water at flow rates of 0.4 μl/min. Mass spectrometry was performed by an ion-trap mass spectrometer (Orbitrap, Thermo) in a positive mode using repetitively full MS scans followed by collision induced dissociation (CID) of the 5 most dominant ions selected from the first MS scan. The mass spectrometric data was clustered and analyzed using the Sequest software (J. Eng and J. Yates, University of Washington and Finnigan, San Jose) and Pep-Miner, searching against the human sequences within the NR-NCBI database.

### Example 1

### RNAi Screen Identifies Atg12 as a Positive Mediator of Apoptosis

To identify individual autophagic genes that function as positive mediators of apoptosis, a library of siRNA pools targeting most of the known mammalian autophagy genes was screened. To this end, HEK293 cells were reverse-transfected with the appropriate siRNAs (siGENOME; Dharmacon) 24 h prior to application of the apoptotic stimulus. Cells were then exposed to the DNA-damaging drug etoposide for a period of 48 h, and the extent of apoptosis was determined by measuring the activity of caspase-3 and -7 using the Caspase-Glo® 3/7 luminescent assay. The screen was performed in three independent biological replicates, each containing three technical replicates per siRNA. Positive mediators of apoptosis were defined as genes whose knockdown led to significant attenuation of caspase-3/7 activity compared to non-targeting controls in all three biological repeats of the screen.

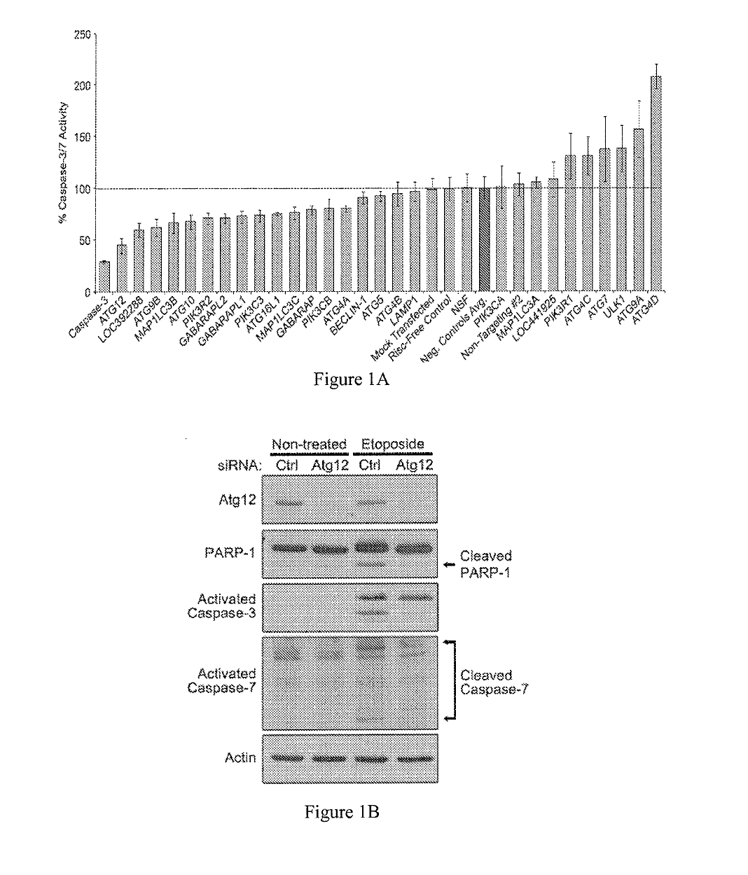

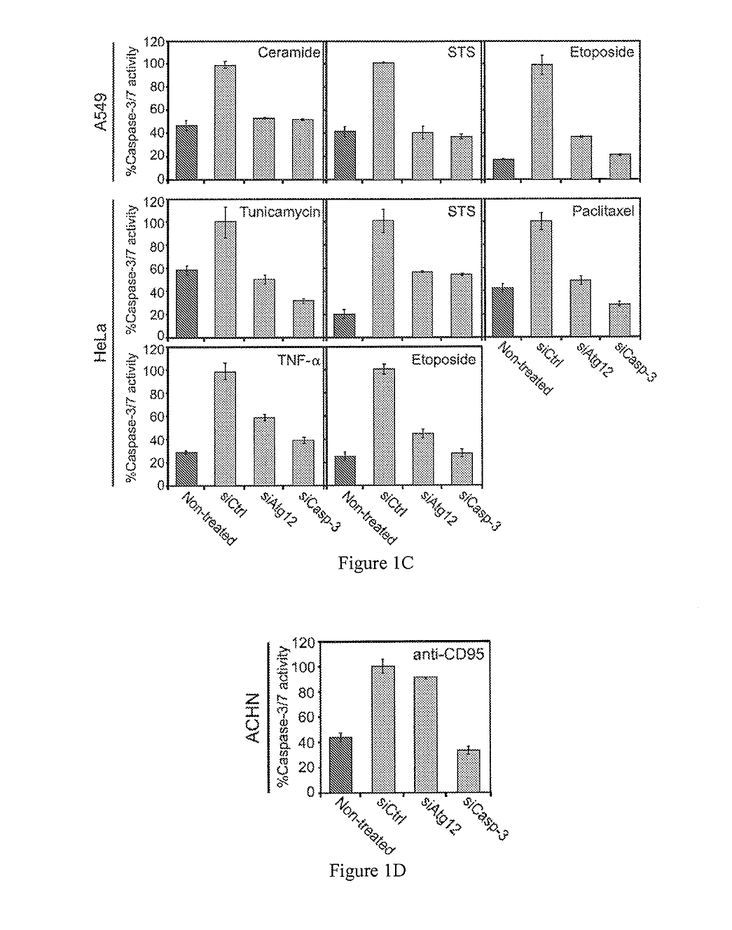

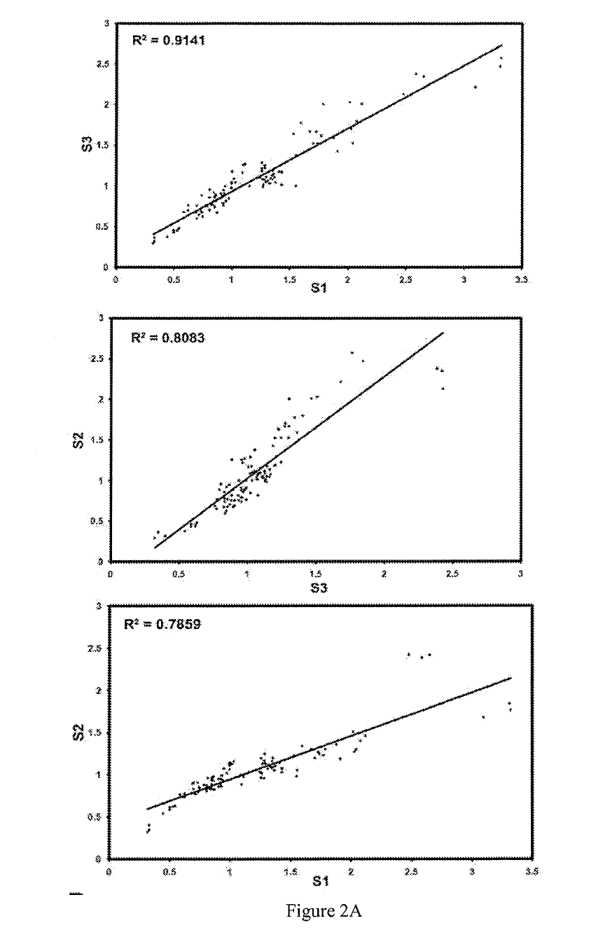

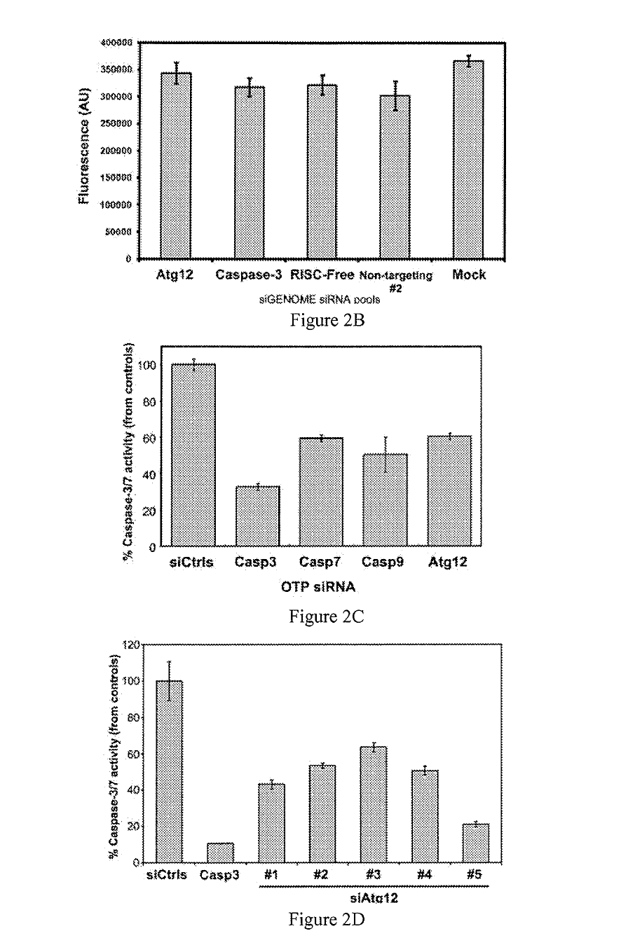

Data obtained from the three biological replicates of the screen exhibited a high degree of correlation (FIG. 2A). Analysis of the screen results according to autophagic gene families revealed that while some families exhibited a uniform behavior, others displayed marked variability in the effect of individual family members on caspase activity (FIG. 1A). For example, knockdown of five out of the six mammalian orthologues of Atg8 (LC3B, LC3C, GABARAP, GABARAPL1,2) led to a moderate decrease in caspase activity, raising the possibility that these proteins may share a similar, and perhaps not fully redundant, function in apoptosis. In contrast to the Atg8 family, the four orthologues of the protease Atg4 (Atg4A-D) displayed substantial variability in their effect on caspase activity, with Atg4A and Atg4C/D having opposite effects. These initial observations require further validation in order to fully elucidate the possible involvement of these gene families in apoptosis. Nevertheless, the finding that some autophagic genes inhibited caspase activity, while others stimulated caspase activity or had no effect, indicates that the observed effects are most likely a result of independent functions of specific autophagy genes in their crosstalk with the apoptosis pathway, rather than a global effect of autophagy inhibition on cell death.

The most pronounced effect, however, was observed upon knockdown of the essential autophagy gene Atg12, which led to a marked inhibition of caspase activity (FIG. 1A, p<0.0001). The depletion of Atg12 was distinct from other autophagy genes, which exhibited milder effects on caspase activity, suggesting that Atg12 was essential for effective progression of apoptosis induced by DNA damage. Similar results were obtained when activation of caspase-3 and -7 was evaluated by western blot analysis under similar conditions. As shown in FIG. 1B, both the processing of caspase-3 and -7 to their active forms as well as cleavage of their downstream target PARP-1 were attenuated in response to knockdown of Atg12 in etoposide-treated cells.

Parallel measurements of DNA content per well indicated that cell number did not vary between Atg12-depleted cells and control siRNA-transfected cells, excluding possible false positive effects due to reduced cell number (FIG. 2B). To address the possibility of off-target effects, the results of the screen were validated by knocking down Atg12 with chemically modified ON-TARGETpIus (OTP) siRNA (Jackson et al., 2006), with similar results as the original screen (FIG. 2C). Further validation was carried out by depletion of Atg12 using five individual siRNA duplexes. All five siRNAs led to a significant attenuation of effector caspase activity in response to etoposide treatment (FIG. 2D).

To verify that the function of Atg12 in apoptosis is not limited to a specific cell type or trigger, the effect of Atg12 depletion was examined in additional cell types and with several different inducers of apoptosis (FIG. 1C). Reduced activity of caspase-3/7 was observed upon knockdown of Atg12 in HeLa cells treated with staurosporine (STS), paclitaxel (taxol), TNF-α, etoposide or tunicamycin; and in A549 cells treated with STS, C₆-ceramide, or etoposide. Knockdown of caspase-3 served as a positive control in these experiments. Notably, the results were reproduced with a second siRNA duplex targeting Atg12. In contrast, depletion of Atg12 had little effect on caspase activation by CD-95 agonistic antibody in the type-I cell lines, ACHN (FIG. 1D) and HepG2. In these cells, Fas-induced apoptosis proceeds via the mitochondria-independent pathways of extrinsic apoptosis (Barnhart et al., 2004). Thus, the effect of Atg12 depletion is limited to apoptotic pathways that converge on mitochondria.

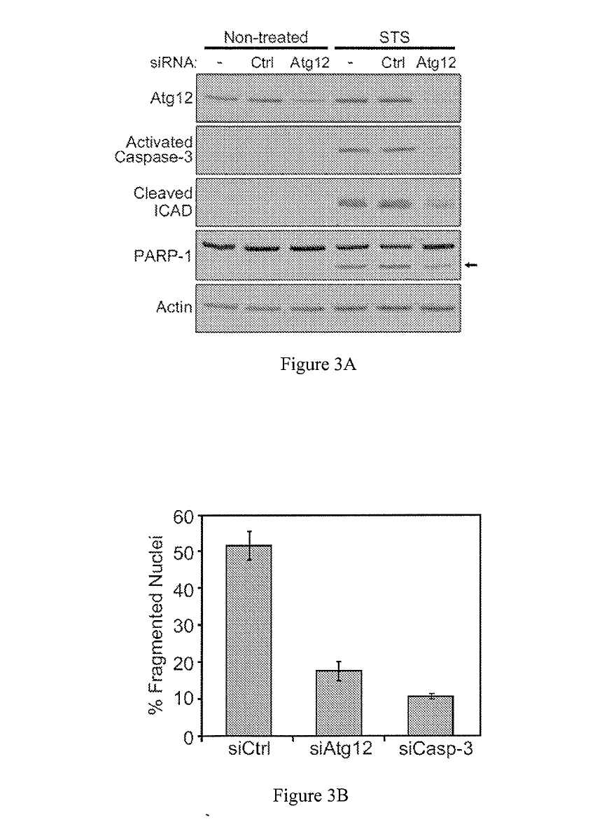

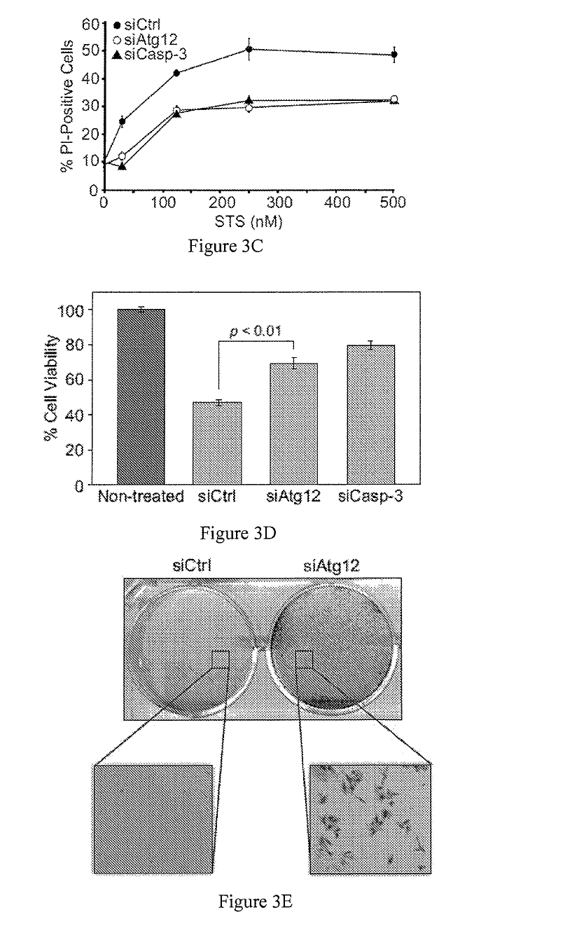

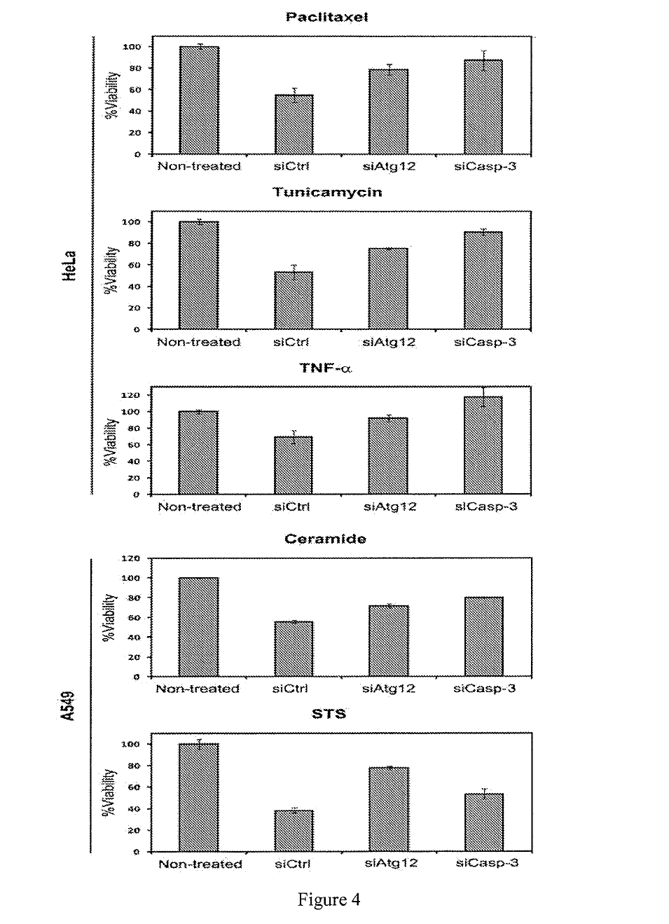

Because the pan-kinase inhibitor STS induces rapid activation of mitochondrial apoptosis, as opposed to other triggers which also activate complex cellular responses such as the DNA damage response or the ER stress response, this trigger was further analyzed in depth. In STS-treated HeLa cells, the attenuation of caspase-3 by Atg12 depletion was accompanied by a decrease in cleavage of its direct downstream substrates, ICAD and PARP-1 (FIG. 3A). At the cellular level, Atg12-depleted cells exhibited decreased morphological signs of nuclear fragmentation, a hallmark of apoptotic cell death (FIG. 3B). More importantly, the impairment in caspase activation upon loss of Atg12 also correlated with decreased susceptibility to STS-induced cell death, as assessed by plasma membrane integrity (FIG. 3C), metabolic capacity (FIG. 3D) and clonogenic survival (FIG. 3E). Increased viability of Atg12-depleted cells was likewise observed with several other apoptotic triggers and cell lines (FIG. 4).

### Example 2

### Atg12 Associates with Bcl-2 Family Members Via a BH3-Like Motif

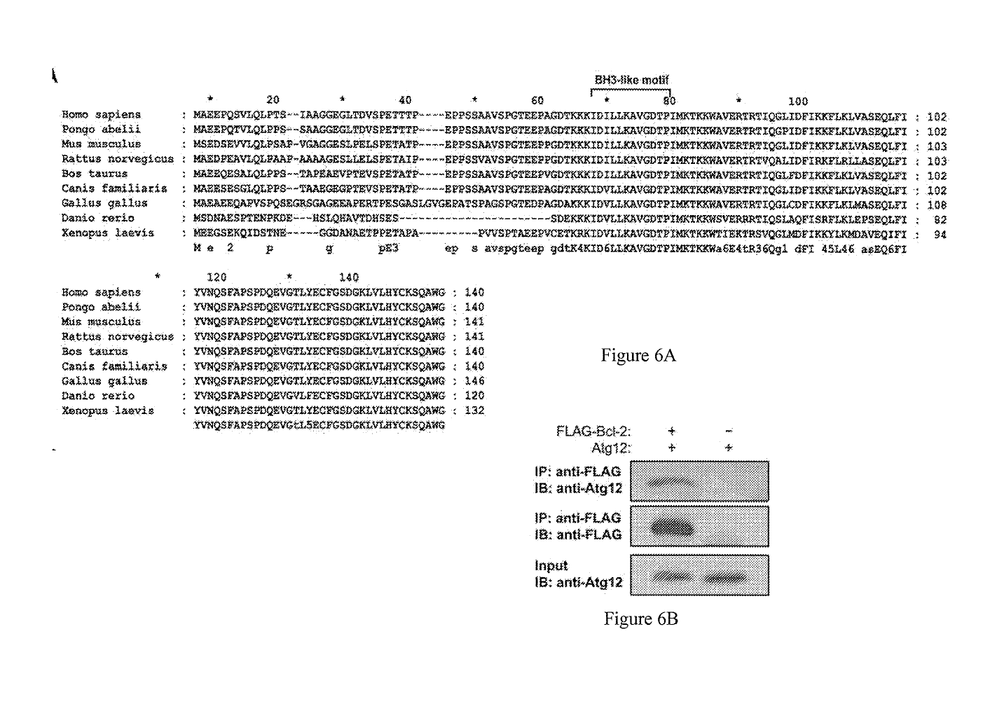

It was hypothesized that one possible way by which Atg12 could regulate apoptosis is via direct interaction with components of the apoptotic pathway. A search for interaction motifs within Atg12 identified a region bearing sequence similarity with BH3 domains (FIG. 5A). Multiple sequence alignment revealed that this ‘BH3-like’ region of Atg12 was conserved among several mammalian and non-mammalian vertebrate species (FIG. 6A). Similar to pro-apoptotic BH3-only proteins of the Bcl-2 family, Atg12 did not contain other BH domains (BH1, BH2 or BH4). However, while BH3 domains assume a prototypical alpha helical fold, the BH3-like motif of Atg12 contains a conserved helix-breaking proline as one of the four residues that typically make up the hydrophobic face of BH3 helices (FIG. 5A). Thus, despite sharing sequence similarity with BH3 domains, the BH3-like region of Atg12 is predicted to differ considerably in its structural fold.

The identification of a BH3-like region suggested that Atg12 may be able to interact with anti-apoptotic Bcl-2 family members. Previous studies have demonstrated that while some BH3-only proteins are able to bind to all Bcl-2 anti-apoptotic proteins, others exhibit selective binding to one of two subgroups of Bcl-2 proteins, the first comprising Bcl-2, Bcl-X_{L} and Bcl-w, and the second comprising Mcl-1 and A1 (Willis and Adams, 2005). Therefore experiments were performed to test whether Atg12 was able to interact with members of each subgroup, specifically Bcl-2 and Mcl-1. In transiently transfected HEK293 cells, wild-type Bcl-2 co-immunoprecipitated with Flag-Atg12 (FIG. 5B). In contrast, point mutation in the BH1 domain of Bcl-2 (Bcl-2 G145A; Yin et al., 1994) completely abolished the interaction with Atg12, indicating that the binding requires the BH3-binding pocket of Bcl-2 (FIG. 5B). The specificity of the interaction was further confirmed by reciprocal co-immunoprecipitation, in which Flag-Bcl-2 pulled down ectopically expressed Atg12 (FIG. 6B). In addition, Mcl-1 also co-immunoprecipitated with Flag-Atg12 upon co-expression (FIG. 5B), and Flag-Atg12 interacted with endogenous Bcl-2 and Mcl-1 (FIG. 5C). Thus, Atg12 is able to bind promiscuously to both subgroups of Bcl-2-related proteins. Finally, endogenous Atg12 co-immunoprecipitated with endogenous Mcl-1 in both HEK293 and HeLa cells, demonstrating that the interaction occurs in physiological conditions and in multiple cell types (FIG. 5D). Interestingly, the binding of Flag-Atg12 to endogenous Bcl-2 was increased in response to STS treatment in HeLa cells, without an apparent increase in protein levels (FIG. 5E), implying that Atg12-Bcl-2 interaction may be regulated post-translationally in response to apoptosis induction.

To further confirm that Atg12 binding occurs within the BH3-binding pocket of Bcl-2 family members, the ability of known Bcl-2 inhibitors to compete out Atg12-Bcl-2 interaction was examined. Indeed, the interaction between Flag-Atg12 and endogenous Bcl-2 was disrupted by co-expression of the BH3-only protein Bad (FIG. 5F), or by incubation of Atg12 immunoprecipitates with ABT-737, a small-molecule BH3 mimetic inhibitor of Bcl-2 (FIG. 5G). Notably, the binding of Atg12 to Mcl-1 was not affected by ABT-737, which specifically targets Bcl-2 (FIG. 5G).

In the yeast Saccharomyces cerevisiae and in Drosophila melanogaster, the BH3-like region of Atg12 deviates from the BH3 consensus motif; namely, a conserved Asp residue found in most BH3 domains (corresponding to D64 in hAtg12) is replaced by Ser in yeast and Asn in flies (FIG. 5A, denoted by arrow). Notably, this Asp is known to be critical for the binding of BH3-only proteins to anti-apoptotic Bcl-2 members. Hence, the sequence variability observed in scAtg12 and dmAtg12 suggested that functionality of the BH3-like region of Atg12 might not extend to these organisms. Indeed, binding of Atg12 to Bcl-2 was severely impaired upon mutation of the conserved D64 in the BH3-like region of hAtg12 to Ser or Asn, resembling the sequences of scAtg12 and dmAtg12, respectively (FIG. 5H). No effect on binding was observed when the conserved D64 was substituted to Ala. This is particularly interesting, as the fly lacks canonical counterparts of BH3-only proteins, and yeast lack homologues of the Bcl-2 family altogether.

Taken together, the data suggest that Atg12 binds to several Bcl-2 family members, and that both the BH3-like region of Atg12 and the BH3-binding groove of Bcl-2 are necessary for the interaction. Notably, although capable of interacting with both subgroups of anti-apoptotic Bcl-2 members, Atg12 did not associate with endogenous or ectopically expressed Bax, a multidomain pro-apoptotic protein of the Bcl-2 family. Thus, in contrast to certain BH3-only proteins, such as tBid, Atg12 is unlikely to induce Bax activation directly.

### Example 3

### Atg12 Interaction with Bcl-2 does not Require Conjugation Activity

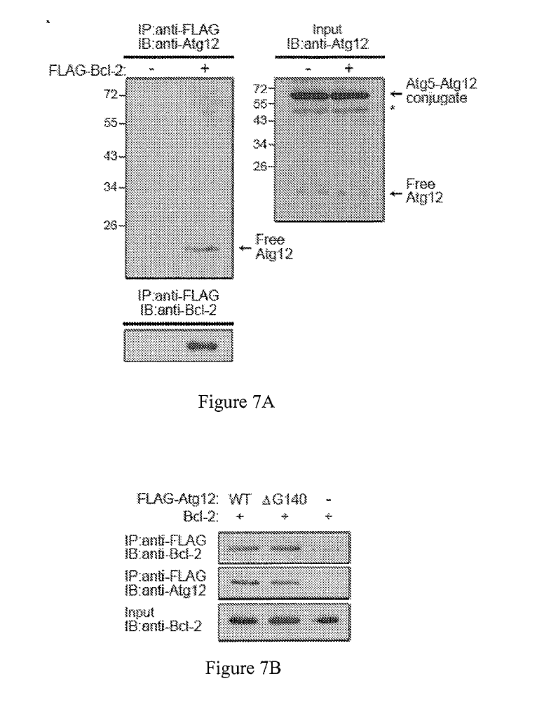

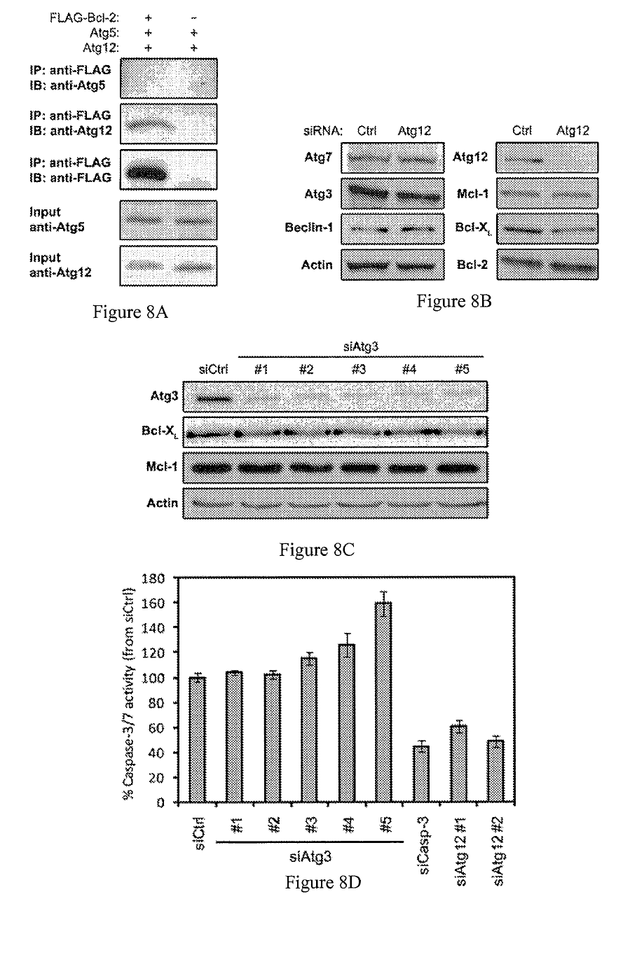

The conjugation of Atg12 to Atg5 is an essential step in the process of autophagy. In order to establish whether Bcl-2 binds to free Atg12, or to the Atg5-Atg12 conjugate, the nature of endogenous Atg12 that is co-immunoprecipitated with Bcl-2 was examined more closely. Upon immunoprecipitation of Flag-tagged Bcl-2 from HEK293 cells, a single band corresponding to the size of unconjugated Atg12 was observed, with no traces at the expected conjugate size (FIG. 7A, left). Furthermore, neither of Atg12's reported conjugation partners, Atg5 or Atg3, could be detected on this blot. Likewise, overexpressed Atg5 did not co-immunoprecipitate with Bcl-2 in transiently transfected HEK293 cells, in agreement with previous observations (Yousefi et al., 2006; FIG. 8D). The interaction between Bcl-2 and free Atg12 is significant, considering that unconjugated Atg12 represents only a small fraction of cellular Atg12, whereas the majority of the protein is conjugated to Atg5, even under basal conditions when autophagy is not induced (FIG. 7A, right).

To further confirm that unconjugated Atg12 is sufficient for interaction with Bcl-2, cells were transfected with a mutant Atg12 lacking the C-terminal Gly that is essential for conjugation with both Atg5 and Atg3 (Atg12ΔG140). The conjugation-defective mutant was able to bind Bcl-2 with the same efficiency as WT Atg12 (FIG. 7B), indicating that the interaction with Bcl-2 family members is mediated exclusively by Atg12, and does not require the ubiquitin-like conjugation machinery.

Recently, disruption of Atg12 conjugation to Atg3 by knockout of Atg3 in mouse embryonic fibroblasts (MEFs) was shown to upregulate levels of Bcl-X_{L}, leading to inhibition of mitochondria-based apoptosis (Radoshevich et al., 2010). In the current experimental system, however, expression levels of Bcl-X_{L} and other anti-apoptotic members of the Bcl-2 family remained unchanged in response to disruption of Atg12-Atg3 conjugation by knockdown of either Atg12 (FIG. 8A), or Atg3 (FIG. 8B). Moreover, and in strong contrast to Atg12, the knockdown of Atg3 with five different siRNAs did not reduce caspase activity in etoposide-treated HEK293 cells (FIG. 84C), or STS-treated HeLa cells. Thus, the outcome of Atg12 depletion on apoptosis reported here cannot be attributed to its conjugation to Atg3.

### Example 4

### Structural Insights into the Mechanism of Atg12-Mcl-1 Interaction by Computational Docking

The presence of a helix-breaking proline in the BH3-like motif of Atg12 suggested that Atg12 may differ considerably from BH3-only proteins in its mode of binding to Bcl-2 family members. To gain insight on this matter, in silico protein-protein docking was used to model the interaction with Bcl-2 family proteins. The structure of hAtg12 was modeled based on plant Atg12 (Suzuki et al., 2005), available from the Protein Data Bank (PDB; Berman et al., 2000). The sequences of plant and human Atg12 exhibit 42% identity along the whole polypeptide chain, rendering the plant structure an adequate modeling template. The crystal structure of plant Atg12 is of a homodimer with domain swapping, and the loop 105-NQSF-108 of the Atg12 monomer was modeled to connect the swapped domains. As predicted from the protein sequence, the BH3-like motif of Atg12 did not form an alpha-helix, but rather a loop comprising residues 62-VGDTPIK-68, of which D64 is involved in binding of Bcl-2. The structure of loop 93-KLVAS-97, which is three residues longer in the modeled sequence, was determined using molecular dynamics. Notably, this loop is spatially adjacent to the BH3-like region.

As both Bcl-2 and Mcl-1 change conformation upon ligand binding, it was expected that the conformer that binds Atg12 would be more similar to the ligand-bound protein than to the free protein. Therefore, the final model of Atg12 was docked to Mcl-1, since a high-resolution structure of this protein with a bound ligand was available.

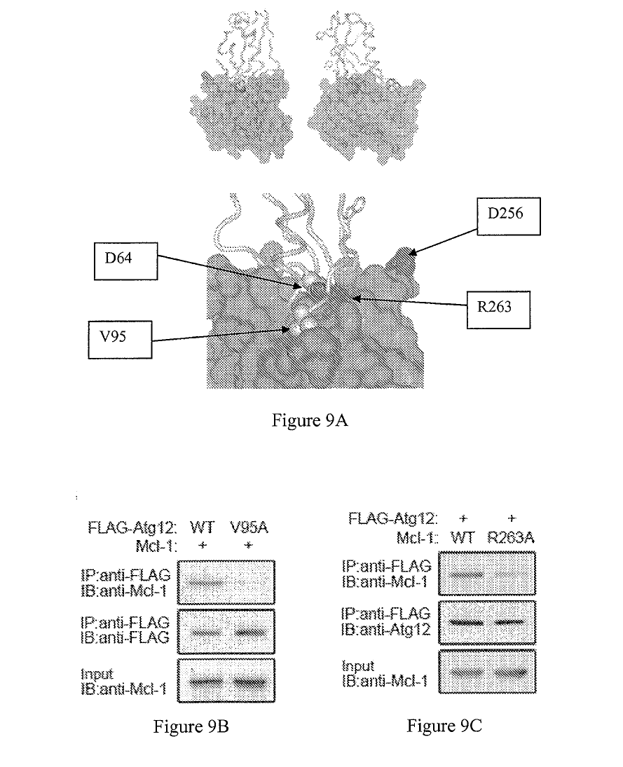

Docking models were generated using a comprehensive geometric-electrostatic-hydrophobic (GEH) scan performed with the program MolFit (Berchanski et al., 2004), followed by a post-scan filter that tests statistical propensity measures and estimates the desolvation energy of the predicted interfaces (Kowalsman and Eisenstein, 2009). 610 models passed this filter. At this point, biological information was introduced, and only models that involved the BH3 binding cavity of Mcl-1 and residue D64 of Atg12 at the interface were retained, producing 14 possible models. The energy-minimized top ranking model is shown in FIG. 9A; According to the model, the intra-molecular ion pair of Mcl-1 R263/D256 is predicted to be broken upon Atg12 binding and replaced by an inter-molecular ion pair with Atg12/D64. The docking model also predicted that additional hydrophobic contacts with the BH3-binding cavity are made by Atg12/V95 from the adjacent loop 93-97.

The hypothetical model was then experimentally tested by mutating the residues in Atg12 and Mcl-1 that were predicted to form crucial contact points. Co-immunoprecipitation experiments indicated that mutation of Atg12/V95 to Ala severely reduced the ability of Atg12 to interact with Mcl-1 (FIG. 9B). Additionally, mutation of Mcl-1/R263, which was predicted to interact with Atg12/D64 from the BH3-like region, resulted in decreased co-immunoprecipitation with Flag-Atg12 (FIG. 9C). In contrast, mutation of residues in Atg12 that were not predicted to contribute to the interaction with Mcl-1, e.g. substitution of F101 (FIG. 10) and T65 to Ala, did not affect levels of co-immunoprecipitated Mcl-1. Thus, the computational model accurately predicted critical contact points between Atg12 and Mcl-1, providing the means to generate mutants for further experimental assessment of the functional significance of this interaction.

### Example 5

### Atg12 Functions Upstream of Mitochondrial Outer Membrane Permeabilization

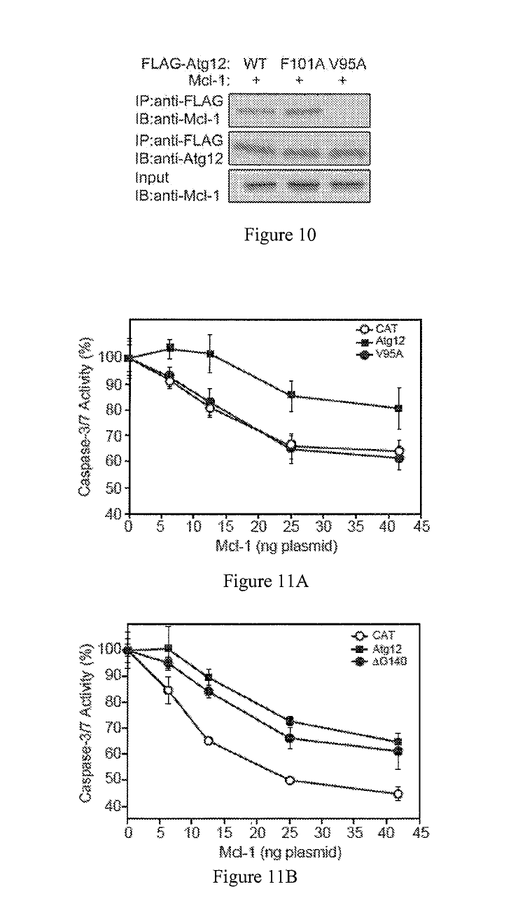

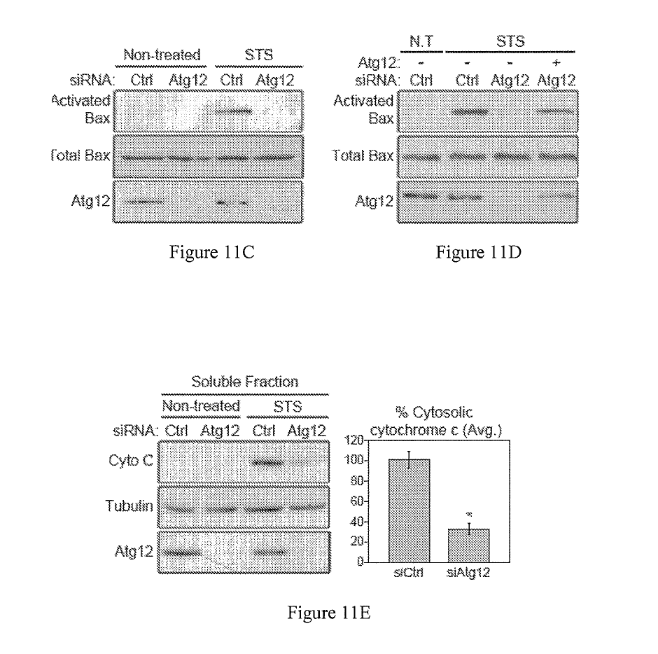

To determine whether Atg12 promotes apoptosis in a similar manner to BH3-only proteins, by antagonizing the function of pro-survival Bcl-2 members, the functional implications of the Atg12-Mcl-1 interaction was assessed. To this end, HeLa cells were transfected with increasing amounts of Mcl-1 plasmid, alone or in combination with Atg12. Expression of Mcl-1 together with a control plasmid (CAT) protected HeLa cells from STS-induced apoptosis in a dose-dependent manner (FIG. 11A). Co-expression of Atg12, however, mitigated the protective effect of Mcl-1, leading to increased caspase activity. Strikingly, the ability of Atg12 to antagonize Mcl-1 was completely abolished when the Mcl-1 binding-defective mutant of Atg12 (V95A) was expressed, suggesting that the pro-apoptotic function of Atg12 is dependent on its ability to bind pro-survival Bcl-2 members. In contrast, the conjugation-defective mutant of Atg12 (ΔG140) behaved similarly to wild-type Atg12, indicating that conjugation to Atg5 or Atg3 is not required for Atg12's ability to suppress Mcl-1 function (FIG. 11B). Notably, overexpression of Atg12 alone was not sufficient to induce apoptosis.

Next, it was assessed whether the depletion of Atg12 affected early events in the apoptotic cascade that are regulated by anti-apoptotic Bcl-2 proteins. In HeLa cells, treatment with STS led to activation of Bax, as determined by a conformation-specific antibody (6A7) that recognizes activated Bax. In contrast, Bax activation was almost completely blocked in response to Atg12 depletion (FIG. 11C). Importantly, Bax activation was restored upon enforced expression of an siRNA-resistant construct of Atg12 (FIG. 11D). Consistent with reduced Bax activation, release of cytochrome c from mitochondria to the cytosol, a key step in the initiation of apoptosis, was also significantly attenuated in Atg12-depleted cells (FIG. 11E). Hence, the apoptotic function of Atg12 lies upstream of mitochondrial outer membrane permeabilization (MOMP), consistent with inhibition of anti-apoptotic Bcl-2-related proteins.

### Example 6

### Bcl-2 Binding-Deficient Mutants of Atg12 Retain Autophagic Function

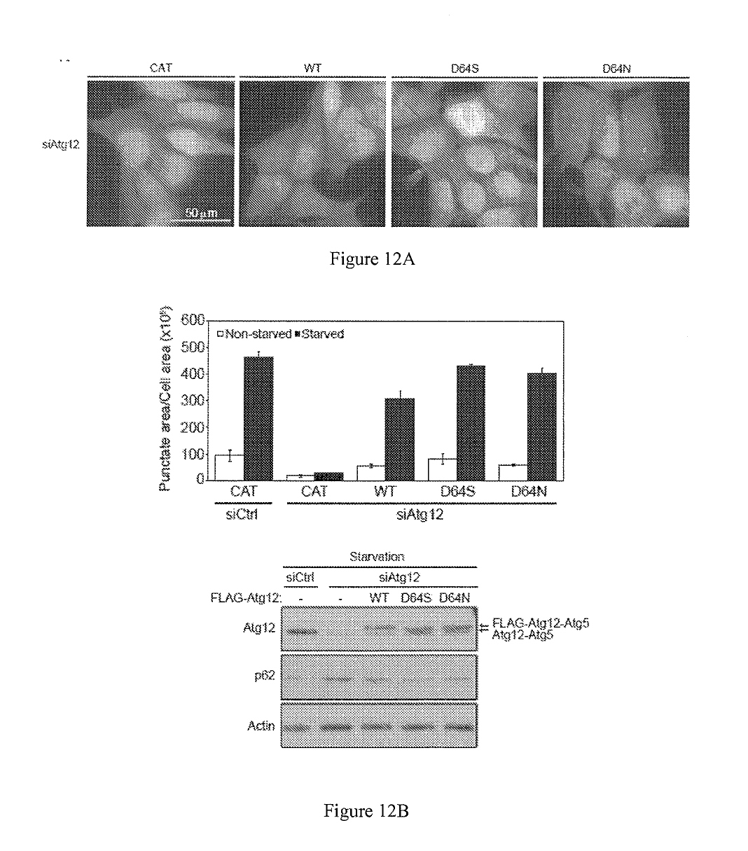

Next, it was assessed whether the mutations in the BH3-like region that abolished the interaction with Bcl-2 also affected the canonical function of Atg12 in autophagy. To this end, reconstitution assays in HEK293 cells stably expressing GFP-LC3 were performed, in which endogenous Atg12 was knocked down by siRNA. In Atg12-depleted cells, ectopic expression of siRNA-resistant constructs of WT Atg12 or the BH3 D64S/N mutants restored levels of the Atg5-Atg12 conjugate (FIG. 12B), indicating that the conjugation process was not impaired by mutations in the BH3-like region. Autophagy levels were then measured by following the distribution pattern of GFP-LC3. In response to induction of autophagy by nutrient deprivation, GFP-LC3 redistributes from diffuse cytoplasmic localization to punctate structures that correspond to autophagosomal membranes, and is thus widely used as a marker for autophagic activity. As expected, in Atg12-depleted cells undergoing starvation, GFP-LC3 was mostly diffuse throughout the cytoplasm, reflecting a defect in autophagosome formation (FIG. 12A). In contrast, GFP-LC3 displayed clear punctate staining when Atg12-depleted cells were reconstituted with either WT Atg12 or the Bcl-2 binding-deficient mutants (FIG. 12A), indicating that autophagy was restored. Similarly, while p62, a specific target of autophagy that is used as a marker for autophagic flux, accumulated in Atg12 depleted cells, its levels were significantly reduced upon re-introduction of either WT Atg12 or the BH3-like mutants. Taken together, the data suggest that the Bcl-2 binding-deficient mutants of Atg12 retain their autophagic function. Thus, the apoptotic and autophagic functions of Atg12 seem to be independent arms of the protein.

### Example 7

### p38 Phosphorylates Atg12

As Atg12 promotes apoptosis by binding and inhibiting Bcl-2, it was reasoned that the Atg12-Bcl-2 interaction is induced in apoptotic cells (and kept in check in non-apoptotic cells). Since no changes were observed in the protein levels of Atg12 or Bcl-2 in response to an apoptotic stimulus, it was reasoned that such regulation would likely occur at the post-translational level, such as phosphorylation of Atg12, Bcl-2 or both.

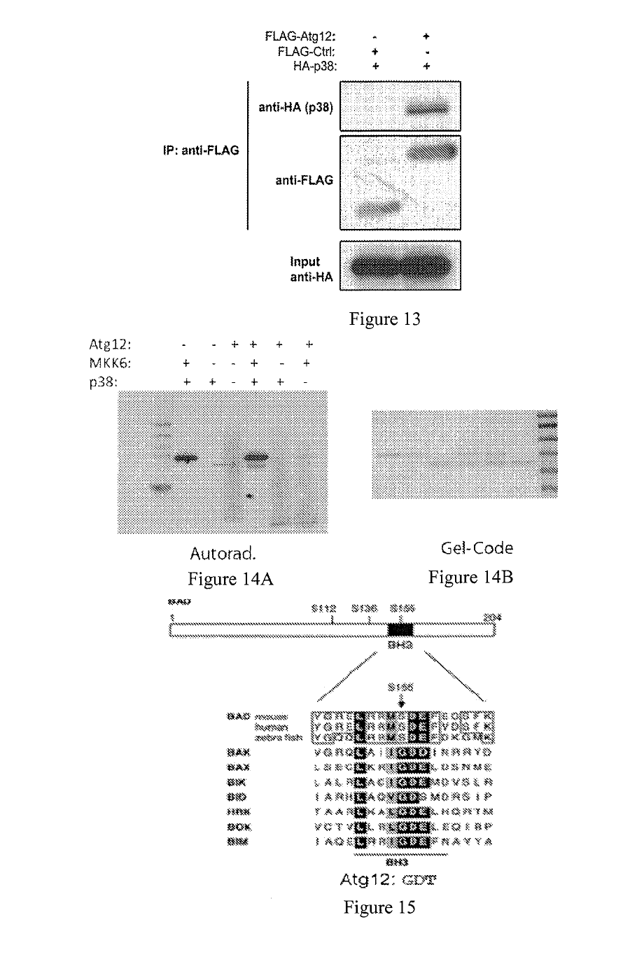

Mining interaction databases, it was discovered that Atg12 is an interacting partner of the kinase p38b in drosophila melanogaster (https://interfly.med.harvard.edu/). This interaction was identified in a large scale high-throughput screen for interacting proteins in drosophila and was given a low confidence score. Based on this finding, the inventors examined whether human Atg12 could interact with the human homolog of p38, specifically p38 alpha, the main isoform of p38 in mammalians (there are four isoforms of this kinase in human). A co-immunoprecipitation experiment using overexpressed proteins (as described in the material and methods section above) revealed that the two proteins indeed interact (FIG. 13).

After validating that Atg12 could interact with p38 in a mammalian system, attempts have been made to determine whether p38 could phosphorylate Atg12. Using recombinant Atg12 and p38 purified from bacteria, it was concluded that p38 phosphorylates Atg12 in an in-vitro kinase assay (FIG. 14A). The technical aspects of this experiment are detailed in the materials and methods section above. In order to identify the specific site of phosphorylation in Atg12, a sample of in-vitro phosphorylated Atg12 was analyzed using mass-spectrometry analysis. The phospho-mapping revealed that the site of phosphorylation was Thr65 in Atg12.

Interestingly, Thr65 is located within the BH3-like domain of Atg12. The finding of the present invention that the BH3-like motif is required for the interaction of Atg12 with Bcl-2 family proteins, together with the finding that Thr65 of Atg12 is phosphorylated by p38, indicates that Thr65 phosphorylation regulates the interaction of Atg12 with Bcl-2 family proteins. Furthermore, a multiple sequence alignment of bona-fide BH3-only proteins revealed that most of these proteins contain a negatively charged residue in the corresponding site (FIG. 15). This indicates that phosphorylation of Atg12's BH3-like motif on Thr65 renders it more similar to canonical BH3-only domains, therefore resulting in enhanced binding to Bcl-2, leading to the initiation of apoptosis. Notably, p38 is known to be a pro-apoptotic kinase activated by diverse apoptotic stimuli.

Of note, p38 was reported by several groups as an inhibitor of autophagy (for example, Webber and Tooze, EMBO J. 2010 Jan. 6; 29(1):27-40). However, to this date, the specific mechanism by which p38 inhibits autophagy remains elusive, and the specific phosphorylation target responsible for this inhibition has not yet been identified. One of the possible implications of the present findings is that inhibition of autophagy by p38 occurs via phosphorylation of Atg12, an indispensable component of the autophagic core machinery.

Lastly, the present invention provides two results of Atg12 phosphorylation, namely 1) phosphorylation of Atg12 leads to apoptosis induction and 2) phosphorylation of Atg12 leads to inhibition of autophagy, which indicates a “molecular switch”. p38 phosphoylation of Atg12 serves to inhibit the autophagic function of Atg12 (thereby inactivating autophagy, a pro-survival pathway) while simultaneously activating Atg12's cell death-promoting function, by enhancing its interaction with Bcl-2. Shutting down a pro-survival pathway while at the same time activating a pro-cell death pathway may allow the cell to achieve a smooth and efficient transition from a pro-survival to a cell death response.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An isolated peptide comprising an amino acid sequence having no more than 30 amino acids as set forth in SEQ ID NO: 1 (IDILLKAVGDTP), or an analog or derivative thereof.

2. The isolated peptide of claim 1, wherein the isolated peptide consists of no more than 25 amino acids, no more than 20 amino acids or no more than 15 amino acids.

3. The isolated peptide of claim 1, wherein the isolated peptide consists of SEQ ID NO: 1.

4. The isolated peptide of claim 1, wherein the analog has at least 70% sequence identity to SEQ ID NO: 1.

5. The isolated peptide of claim 4, wherein the analog has the amino acid sequence selected from the group consisting of: SEQ ID NO: 3 (IDVLLKAVGDTP), SEQ ID NO: 5 (IDVLLKAVGDDP) and SEQ ID NO: 6 (IDVLLKAVGDEP).

6. The isolated peptide of claim 1, wherein said peptide binds a Bcl-2 anti-apoptotic protein, or wherein said peptide is a pro-apoptotic peptide.

7. The isolated peptide of claim 6, wherein said peptide binds the BH3 binding pocket of the Bcl-2 anti-apoptotic protein.

8. The isolated peptide of claim 6, wherein the Bcl-2 anti-apoptotic protein is selected from the group consisting of: Bcl-2, Bcl-X_{L}, Bcl-w, Bcl-B, Mcl-1 and A1 (Bfl-1).

9. The isolated peptide of claim 8, wherein the Bcl-2 anti-apoptotic protein is Bcl-2 or Mcl-1.

10. A pharmaceutical composition comprising the isolated peptide of claim 1 as an active ingredient, and a pharmaceutically acceptable carrier.

11. A method for inducing apoptosis in a cell, the method comprising contacting the cell with the peptide of claim 1.

12. The method of claim 11, wherein said cell is a cancer cell.

13. A method for treating cancer in a subject in need thereof, the method comprising administering to the subject a therapeutically effective amount of a pharmaceutical composition according to claim 10.

14. The method of claim 13, wherein the peptide sensitizes cancer cells to chemotherapy treatment or wherein the method further comprises administering to the subject a chemotherapeutic agent.

15. The method of claim 14, wherein the chemotherapeutic agent is an apoptosis-inducing chemotherapeutic agent or wherein the cancer is selected from a hematopoietic malignancy, a solid malignancy and a chemoresistant cancer.

16. The method of claim 15, wherein the cancer is a hematopoietic malignancy selected from the group consisting of: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma and non-Hodgkin's lymphoma or wherein the cancer is a solid malignancy selected from the group consisting of: prostate cancer, breast cancer, skin cancer, colon cancer, lung cancer, pancreatic cancer, head and neck cancer, kidney cancer, ovarian cancer, cervix cancer, bone cancer, liver cancer, thyroid cancer and brain cancer.

17. The method according claim 13, wherein the subject is a mammal or a human.

18. A method of screening for a pro-apoptotic agent, the method comprising the steps of:
exposing a cell expressing Atg12 and a Bcl-2 anti-apoptotic protein to a putative pro-apoptotic agent; and
determining the binding of Atg12 to the Bcl-2 anti-apoptotic protein;
wherein enhancement of said binding indicates that the agent is a pro-apoptotic agent.

19. The method of claim 18, further comprising the step of:
determining the change in survival of the cell in the presence of the agent relative to a control.

20. The method of claim 18, wherein the Bcl-2 anti-apoptotic protein is selected from the group consisting of: Bcl-2, Bcl-X_{L}, Bcl-w, Bcl-B, Mcl-1 and A1 (Bfl-1) and wherein the putative agent is selected from the group consisting of: peptides, nucleic acids, organic molecules, inorganic compounds and antibodies or antigen binding fragments thereof.

21. A pro-apoptotic agent obtained by the method according to claim 18.

22. A method of screening for an anti-apoptotic agent, the method comprising the steps of:
exposing a cell expressing Atg12 and a Bcl-2 anti-apoptotic protein to a putative anti-apoptotic agent; and
determining the binding of Atg12 to the Bcl-2 anti-apoptotic protein;
wherein reduction of said binding indicates that the agent is an anti-apoptotic agent.

23. The method of claim 22 further comprising:
determining the change in survival of the cell in the presence of the agent relative to a control, or wherein the putative agent is selected from the group consisting of: peptides, nucleic acids, organic molecules, inorganic compounds and antibodies or antigen binding fragments thereof.

24. An anti-apoptotic agent obtained by the method according to claim 22 or a pharmaceutical composition comprising said anti-apoptotic agent as an active ingredient, and a pharmaceutically acceptable carrier.

25. A method for treating a disease or disorder characterized by excessive cellular death in a subject in need thereof, the method comprising administering to the subject a therapeutically effective amount of the anti-apoptotic agent of claim 24.

26. The method of claim 25, wherein the disease or disorder characterized by excessive cellular death is a neurodegenerative disease or wherein the disease or disorder is associated with neuronal cell death.

27. The method of claim 26, wherein the disease or disorder is a neurodegenerative disease selected from the group consisting of: Alzheimer's disease, Huntington's disease, Parkinson's disease, neurodegeneration due to stroke, amyotrophic lateral sclerosis (ALS), Pick's disease, Progressive Supranuclear Palsy (PSP), fronto-temporal dementia (FTD), pallido-ponto-nigral degeneration (PPND), Guam-ALS syndrome, pallido-nigro-luysian degeneration (PNLD) and cortico-basal degeneration (CBD), or wherein the disease or disorder is associated with neuronal cell death and is selected form the group consisting of: epilepsy, hypoxia/ischemia related acute brain injury, Parkinson's disease and Alzheimer's disease.
